(19) United States
(12) Patent Application Publication
(10) Pub. No.: US 2023/0338562 A1
(43) Pub. Date: Oct. 26, 2023

(54) METHODS FOR THE PREPARATION OF LINKER PAYLOAD CONSTRUCTS

(71) Applicant: SYNAFFIX B.V., Oss (NL)

(72) Inventors: Jorge Merijn Mathieu VERKADE, Eindhoven (NL); Sander Sebastiaan VAN BERKEL, Oss (NL); Floris Louis VAN DELFT, Nijmegen (NL)

(21) Appl. No.: 18/087,678

(22) Filed: Dec. 22, 2022

Related U.S. Application Data
(63) Continuation of application No. PCT/EP2021/067754, filed on Jun. 28, 2021.

(30) Foreign Application Priority Data
Jun. 26, 2020 (EP) 20182748.2

(51) Int. Cl. A61K 47/68; A61K 47/54

(52) U.S. Cl. CPC A61K 47/6817; A61K 47/54

(57) ABSTRACT

The present invention concerns a method for the preparation of an alkyne-linker-payload construct of structure Q-L-C(O)—NR³-D (1), comprising reacting (i) an alkyne compound of structure Q-L-C(O)-X (2), wherein Q is an alkyne moiety selected from the group consisting of terminal alkyne and (hetero)cycloalkyne; L is a linker, and X is a leaving group selected from halogen, SR¹, O-succinimidyl, O-(hetero)aryl(R²)₁₋₅, wherein R¹ is selected from C₁-C₆ alkyl and (hetero)aryl; and R² is C₁-C₆ alkyl, halogen or NO₂; with (ii) a molecule of structure D-NHR³ (3), wherein D is a payload, and R³ is selected from hydrogen, optionally substituted C₁-C₂₄ alkyl, optionally substituted aryl. The activated ester derivatives (2) are highly stable and provide for smooth and high-yielding attachment to a cytotoxic payload. The invention further concerns a method for preparing bioconjugates and alkyne compound of structure Q-L-C(O)-X (2).

### CROSS-REFERENCE TO RELATED APPLICATIONS

This application is a continuation of International Patent Application No. PCT/EP2021/067754 filed Jun. 28, 2021, which application claims priority to European Patent Application No. 20182748.2 filed Jun. 26, 2020, the contents of which are both incorporated herein by reference in their entireties.

### FIELD OF THE INVENTION

The present invention relates to the field of bioconjugation, in particular to activated ester derivatives of alkyne compounds which are useful intermediates in the preparation of bioconjugates.

### BACKGROUND OF THE INVENTION

Antibody-drug conjugates (ADC), considered as magic bullets in therapy, are comprised of an antibody to which is attached a pharmaceutical agent. The antibodies (also known as ligands) can be small protein formats (scFv's, Fab fragments, DARPins, Affibodies, etc.) but are generally monoclonal antibodies (mAbs) which have been selected based on their high selectivity and affinity for a given antigen, their long circulating half-lives, and little to no immunogenicity. Thus, mAbs as protein ligands for a carefully selected biological receptor provide an ideal delivery platform for selective targeting of pharmaceutical drugs. For example, a monoclonal antibody known to bind selectively with a specific cancer-associated antigen can be used for delivery of a chemically conjugated cytotoxic agent to the tumour, via binding, internalization, intracellular processing and finally release of active catabolite. The cytotoxic agent may be small molecule toxin, a protein toxin or other formats, like oligonucleotides. As a result, the tumour cells can be selectively eradicated, while sparing normal cells which have not been targeted by the antibody. Similarly, chemical conjugation of an antibacterial drug (antibiotic) to an antibody can be applied for treatment of bacterial infections, while conjugates of anti-inflammatory drugs are under investigation for the treatment of autoimmune diseases and for example attachment of an oligonucleotide to an antibody is a potential promising approach for the treatment of neuromuscular diseases. Hence, the concept of targeted delivery of an active pharmaceutical drug to a specific cellular location of choice is a powerful approach for the treatment of a wide range of diseases, with many beneficial aspects versus systemic delivery of the same drug.

An alternative strategy to employ monoclonal antibodies for targeted delivery of a specific protein agent is by genetic fusion of the latter protein to one (or more) of the antibody's termini, which can be the N-terminus or the C-terminus on the light chain or the heavy chain (or both). In this case, the biologically active protein of interest, e.g. a protein toxin like Pseudomonas exotoxin A (PE38) or an anti-CD3 single chain variable fragment (scFv), is genetically encoded as a fusion to the antibody, possibly but not necessarily via a peptide spacer, so the antibody is expressed as a fusion protein. The peptide spacer may contain a protease-sensitive cleavage site, or not.

In the field of ADCs, a chemical linker is typically employed to attach a pharmaceutical drug to an antibody. This linker needs to possess a number of key attributes, including the requirement to be stable in plasma after drug administration for an extended period of time. A stable linker enables localization of the ADC to the projected site or cells in the body and prevents premature release of the payload in circulation, which would indiscriminately induce undesired biological response of all kinds, thereby lowering the therapeutic index of the ADC.

Upon internalization, the ADC should be processed such that the payload is effectively released so it can bind to its target.

There are two families of linkers, non-cleavable and cleavable. Non-cleavable linkers consist of a chain of atoms between the antibody and the payload, which is fully stable under physiological conditions, irrespective of which organ or biological compartment the antibody-drug conjugate resides in. As a consequence, liberation of the payload from an ADC with a non-cleavable linker relies on the complete (lysosomal) degradation of the antibody after internalization of the ADC into a cell. As a consequence of this degradation, the payload will be released, still carrying the linker, as well as a peptide fragment and/or the amino acid from the antibody the linker was originally attached to. Cleavable linkers utilize an inherent property of a cell or a cellular compartment for selective release of the payload from the ADC, which generally leaves no trace of linker after metabolic processing. For cleavable linkers, there are three commonly used mechanisms: 1) susceptibility to specific enzymes, 2) pH-sensitivity, and 3) sensitivity to redox state of a cell (or its microenvironment).

Enzyme-based strategies are generally based on the endogenous presence of specific proteases, esterases, glycosidases or others. For example, the majority of ADCs used in oncology utilize the dominant proteases found in a tumour cell lysosome for recognition and cleavage of a specific peptide sequence in the linker. Dubowchik et al., Bioconjug Chem. 2002, 13, 855-69, incorporated by reference, pioneered the discovery of specific dipeptides as an intracellular cleavage mechanism by cathepsins. Other enzymes that are known to be upregulated in the tumour lysozyme or the tumour microenvironment are plasmin, matrix metalloproteases (MMPs), urokinase, and others, all of which may recognize a specific peptide sequence in the ADCs and induce release of payload from the linker by hydrolytic cleavage of one of the peptide bonds. Other endogenous enzymes that may be employed for tumour-specific hydrolytic cleavage of bonds are for example esterases, glucosidases, phosphatases or sulfatases.

The acid-sensitivity strategy takes advantage of the lower pH in the endosomal (pH 5-6) and lysosomal (˜pH 4.8) compartments, as compared to the cytosol of a human cell (pH 7.4), to trigger hydrolysis of an acid labile group within the linker, such as a hydrazone, see for example Ritchie et al, mAbs 2013, 5, 13-21, incorporated by reference. Alternative acid-sensitive linker may also be employed, as for example based on silyl ethers, disclosed in US20180200273.

A third release strategy based on redox mechanisms exploits the higher concentrations of intracellular glutathione than in the plasma. Thus, linkers containing a disulfide bridge release a free thiol group upon reduction by glutathione, which may remain part of the payload or further self-immolate to release the free payload. Alternative reduction mechanisms for release of free payload can be based on the conversion of an (aromatic) nitro group or a (aromatic) azido group into an aniline, which may be part of a payload or part of a self-immolative assembly unit.

A self-immolative assembly unit in an antibody-drug conjugate links a drug unit to the remainder of the conjugate or its drug-linker intermediate. The main function of the self-immolative assembly unit is to conditionally release free drug at the site targeted by the ligand unit. The activatable self-immolative moiety comprises an activatable group and a self-immolative spacer unit. Upon activation of the activatable group, for example by enzymatic conversion of an amide group to an amino group or by reduction of a disulfide to a free thiol group, a self-immolative reaction sequence is initiated that leads to release of free drug by one or more of various mechanisms, which may involve (temporary) 1,6-elimination of a p-aminobenzyl group to a p-quinone methide, optionally with release of carbon dioxide and/or followed by a second cyclization release mechanism. The self-immolative assembly unit can part of the chemical spacer connecting the antibody and the payload (via the functional group).

Alternatively, the self-immolative group is not an inherent part of the chemical spacer but branches off from the chemical spacer connecting the antibody and the payload.

A self-immolative unit in most cases at least exists of an (acylated) para-aminobenzyl unit connected to a protease-sensitive peptide fragment for enzymatic release of the amino group. Besides the aminobenzyl group, other aromatic moieties may also be employed as part for the self-immolative unit, for example heteroaromatic moieties such as pyridine or thiazoles, see for example U.S. Pat. No. 7,754,681 and US2005/0256030. Substitution of the aminobenzyl group may be in the para position or in the ortho position, in both cases leading the same 1,6-elimination mechanism. The benzylic position may be substituted with alkyl or carbonyl derivatives, for example esters or amides derived from mandelic acid, as for example disclosed in WO2015/038426, incorporated by reference. The benzylic position of the self-immolative unit is connected to a heteroatom leaving group, typically based on, but not limited to, oxygen or nitrogen. Predominantly, the benzylic functional group exists of a carbamate moiety, which will release carbon dioxide upon triggering of the 1,6-elimination mechanism, and a primary or secondary amino group. The primary or secondary amino group may be part of the toxic payload itself and may be an aromatic amino group or an aliphatic amino group. In the latter case, the amino group of the liberated payload will most likely have a pKa higher than and therefore be mostly in a protonated state at physiological conditions (pH 7-7.5), and specifically in the acidic environment of the tumour (pH<7).

The primary or secondary amino group may also be part of another self-immolative group, for example an N,N-dialkylethylenediamine moiety. The N,N-dialkylethylenediamine moiety at the other may be connected to another carbamate group to liberate, upon cyclization, an alcohol group as part of the toxic payload, as for example demonstrated by Elgersma et al, Mol. Pharm. 2015, 12, 1813-1835, incorporated by reference. The primary or secondary amino group of the carbamate moiety may also form part of an N,O-acetal, a method which has been used in several drug delivery strategies, for example to release 5-fluorouracil (Madec-Lougerstay et al, J. Chem. Soc. Perkin Trans I, 1999, 1369-1375) and SN-38 (Santi et al, J. Med. Chem. 2014, 57, 2303-2314). Most recently, a similar configuration was employed by Kolakowski et al, Angew. Chem. Int. Ed. 2016, 55, 7948-7951, incorporated by reference, for design of linkers with prolonged serum exposure because of the long circulation time of ADCs, in combination with a beta-glucuronidase-promoted release mechanism, to release aliphatic alcohols. The functional group at the benzylic position of the self-immolative aromatic moiety may also be a phenolic oxygen, see for example Toki et al, J. Org. Chem. 2002, 67,1866-1872 and U.S. Pat. No. 7,553,816, incorporated by reference, however not an aliphatic alcohol because an aliphatic alcohol does not possess sufficient leaving group capacity (typical pKa 13-15). Another option for the benzylic functional group is a quaternary ammonium group, which will release a trialkylamino group or a heteroaryl amine upon elimination, as reported by Burke et al, Mol. Cancer Ther. 2016, 15, 938-945 and Staben et al, Nat. Chem. 2016, 8, 1112-1119, incorporated by reference.

Currently, payloads utilized in ADCs primarily include microtubule-disrupting agents [e.g. monomethyl auristatin E (MMAE) and monomethyl auristatin F (MMAF) and maytansinoid-derived DM1 and DM4], DNA-damaging agents [e.g., calicheamicin, pyrrolobenzodiazepines (PBD) dimers, indolinobenzodiapines dimers, duocarmycins, anthracyclins], topoisomerase inhibitors [e.g. SN-38, exatecan and derivatives thereof, simmitecan] or RNA polymerase II inhibitors [e.g. amanitin]. Although ADCs have demonstrated clinical and preclinical activity, it has been unclear what factors determine such potency in addition to antigen expression on targeted tumour cells. For example, drug:antibody ratio (DAR), ADC-binding affinity, potency of the payload, receptor expression level, internalization rate, trafficking, multiple drug resistance (MDR) status, and other factors have all been implicated to influence the outcome of ADC treatment in vitro. In addition to the direct killing of antigen-positive tumour cells, ADCs also have the capacity to kill adjacent antigen-negative tumour cells: the so-called “bystander killing” effect, as originally reported by Sahin et al, CancerRes. 1990, 50, 6944-6948 and for example studied by Li et al, Cancer Res. 2016, 76, 2710-2719. Generally spoken, cytotoxic payloads that are neutral will show bystander killing whereas ionic (charged) payloads do not, as a consequence of the fact that ionic species do not readily pass a cellular membrane by passive diffusion. For example, evaluation of a range of exatecan derivatives indicated that acylation of the primary amine with hydroxyacetic acid provided a derivative (DXd) with substantially enhanced bystander killer versus various aminoacylated exatecan derivatives, as disclosed by Ogitani et al, Cancer Sci. 2016, 107, 1039-1046, incorporated by reference.

ADCs are prepared by conjugation of a linker-drug with a protein, a process known as bioconjugation. Many technologies are known for bioconjugation, as summarized in G.T. Hermanson, “Bioconjugate Techniques”, Elsevier, 3^{rd} Ed. 2013, incorporated by reference. Two main technologies can be recognized for the preparation of ADCs by random conjugation, either based on acylation of lysine side-chain or based on alkylation of cysteine side-chain. Acylation of the Q-amino group in a lysine side-chain is typically achieved by subjecting the protein to a reagent based on an activated ester or activated carbonate derivative, for example SMCC is applied for the manufacturing of Kadcyla®. Main chemistry for the alkylation of the thiol group in cysteine side-chain is based on the use of maleimide reagents, as is for example applied in the manufacturing of Adcetris®. Besides standard maleimide derivatives, a range of maleimide variants are also applied for more stable cysteine conjugation, as for example demonstrated by James Christie et al., J. Contr. Rel. 2015, 220, 660-670 and Lyon et al., Nat. Biotechnol. 2014, 32, 1059-1062, both incorporated by reference. Other approaches for cysteine alkylation involve for example nucleophilic substitution of haloacetamides (typically bromoacetamide or iodoacetamide), see for example Alley et al., Bioconj. Chem. 2008, 19, 759-765, incorporated by reference, or various approaches based on nucleophilic addition on unsaturated bonds, such as reaction with acrylate reagents, see for example Bernardim et al., Nat. Commun. 2016, 7, DOI: 10.1038/ncomms13128 and Ariyasu et al., Bioconj. Chem. 2017, 28, 897-902, both incorporated by reference, reaction with phosphonamidates, see for example Kasper et al., Angew. Chem. Int. Ed. 2019, 58, 11625-11630, incorporated by reference, reaction with allenamides, see for example Abbas et al., Angew. Chem. Int. Ed. 2014, 53, 7491-7494, incorporated by reference, reaction with cyanoethynyl reagents, see for example Kolodych et al., Bioconj. Chem. 2015, 26, 197-200, incorporated by reference, reaction with vinylsulfones, see for example Gil de Montes et al., Chem. Sci. 2019, 10, 4515-4522, incorporated by reference, or reaction with vinylpyridines, see for example https://iksuda.com/science/permalink/(accessed Jan. 7^{th}, 2020). An alternative approach to antibody conjugation without reengineering of antibody involves the reduction of interchain disulfide bridges, followed addition of a payload attached to a cysteine cross-linking reagent, such as bis-sulfone reagents, see for example Balan et al., Bioconj. Chem. 2007, 18, 61-76 and Bryant et al., Mol. Pharmaceutics 2015, 12, 1872-1879, both incorporated by reference, mono- or bis-bromomaleimides, see for example Smith et al., J. Am. Chem. Soc. 2010, 132, 1960-1965 and Schumacher et al., Org. Biomol. Chem. 2014, 37, 7261-7269, both incorporated by reference, bis-maleimide reagents, see for example WO2014114207, bis(phenylthio)maleimides, see for example Schumacher et al., Org. Biomol. Chem. 2014, 37, 7261-7269 and Aubrey et al., Bioconj. Chem. 2018, 29, 3516-3521, both incorporated by reference, bis-bromopyridazinediones, see for example Robinson et al., RSC Advances 2017, 7, 9073-9077, incorporated by reference, bis(halomethyl)benzenes, see for example Ramos-Tomillero et al., Bioconj. Chem. 2018, 29, 1199-1208, incorporated by reference or other bis(halomethyl)aromatics, see for example WO2013173391. Typically, ADCs prepared by cross-linking of cysteines have a drug-to-antibody loading of ˜4 (DAR4). Another useful technology for conjugation to a cysteine side-chain is by means of disulfide bond, a bioactivatable connection that has been utilized for reversibly connecting protein toxins, chemotherapeutic drugs, and probes to carrier molecules (see for example Pillow et al., Chem. Sci. 2017, 8, 366-370, incorporated by reference).

Besides conjugation to lysine or cysteine, a range of other conjugation technologies has been explored in the past decade. One method is based on genetic encoding of a non-natural amino acid, e.g. p-acetophenylalanine suitable for oxime ligation, or p-azidomethylphenylalanine suitable for click chemistry conjugation, as for example demonstrated by Axup et al. Proc. Nat. Acad. Sci. 2012, 109, 16101-16106, incorporated by reference. Similarly, Zimmerman et al., Bioconj. Chem. 2014, 25, 351-361, incorporated by reference have employed a cell-free protein synthesis method to introduce azidomethylphenylalanine (AzPhe) into monoclonal antibodies for conversion into ADC by means of metal-free click chemistry. Also, it has also be shown by Nairn et al., Bioconj. Chem. 2012, 23, 2087-2097, incorporated by reference, that a methionine analogue like azidohomoalanine (Aha) can be introduced into protein by means of auxotrophic bacteria and further converted into protein conjugates by means of (copper-catalysed) click chemistry. Finally, genetic encoding of aliphatic azides in recombinant proteins using a pyrrolysyl-tRNA synthetase/tRNAcuA pair was shown by Nguyen et al., J. Am. Chem. Soc. 2009, 131, 8720-8721, incorporated by reference and labelling was secured by click chemistry. The latter method should also be applicable to produce DAR2 ADCs, similar to the method reported by Oller-Salvia et al., Angew. Chem. Int. Ed. 2018, 57, 2831-2834.

Another method is based on enzymatic installation of a non-natural functionality. For example, Lhospice et al., Mol. Pharmaceut. 2015, 12, 1863-1871, incorporated by reference, employ the bacterial enzyme transglutaminase (BTG or TGase) for installation of an azide moiety onto an antibody. A genetic method based on C-terminal TGase-mediated azide introduction followed by conversion in ADC with metal-free click chemistry was reported by Cheng et al., Mol. Cancer Therap. 2018, 17, 2665-2675, incorporated by reference.

It has been shown in WO2014065661, by van Geel et al., Bioconj. Chem. 2015, 26, 2233-2242 and Verkade et al., Antibodies 2018, 7, 12, all incorporated by reference, that enzymatic remodelling of the native antibody glycan at N297 enables introduction of an azido-modified sugar, suitable for attachment of cytotoxic payload using click chemistry.

Chemical approaches have also been developed for site-specific modification of antibodies without prior genetic modification, as for example highlighted by Yamada and Ito, ChemBioChem. 2019, 20, 2729-2737.

Chemical conjugation by affinity peptide (CCAP) for site-specific modification has been developed by Kishimoto et al., Bioconj. Chem. 2019, by using a peptide that binds with high affinity to human IgG-Fc, thereby enabling selective modification of a single lysine in the Fc-fragment with a biotin moiety or a cytotoxic payload. Similarly, Yamada et al., Angew. Chem.

Int. Ed. 2019, 58, 5592-5597, and Matsuda et al., ACS Omega 2019, 4, 20564-20570, both incorporated by reference, have demonstrated that a similar approach (AJICAP™ technology) can be applied for the site-specific introduction of thiol groups on a single lysine in the antibody heavy chain. CCAP or AJICAP™ technology may also be employed for the introduction of azide groups or other functionalities.

Bioconjugation of linker-drugs to azido-modified proteins can be achieved by copper-catalysed alkyne-azide click chemistry (CuAAC) or metal-free, strain-promoted alkyne-azide cycloaddition (SPAAC). In a CuAAC reaction, a linker-drug functionalized with a terminal acetylene is attached to the azido-modified protein under the action of a catalytic amount of copper(I). In a SPAAC reaction, the linker-drug is functionalized with a cyclic alkyne and the cycloaddition is driven by relief of ring-strain, which does not require the presence of a metal additive. Various strained alkynes suitable for metal-free click chemistry are indicated in FIG. 1. Suitable reactive groups for reaction with strained alkynes are exemplified in FIG. 2.

The present invention provides an improved approach towards the synthesis of these alkyne-linker-drugs constructs, which offers highly stable intermediates and a smooth and high-yielding preparation of alkyne-linker-drugs constructs and bioconjugates.

### SUMMARY OF THE INVENTION

The present inventors have found that activated ester derivatives of alkyne compounds unexpectedly facilitate the covalent attachment of the alkyne compound to an amine-functionalized payload molecule. The activated ester derivatives can be conveniently prepared by activation of precursor carboxylic acid, and are (a) highly stable and (b) provide for smooth and high-yielding attachment to a cytotoxic payload, with or without a cleavable linker. As such, the activated ester compounds of the present invention are ideal intermediates in the preparation of bioconjugates, wherein a biomolecule is covalently attached to a cytotoxic payload. The activated ester derivative is first attached to the payload, and subsequently the alkyne moiety is reacted with an appropriately functionalized biomolecule.

The inventors realized that a suitable method to chemically connect two molecules is, i.e. amide bond formation, provides unexpected advantageous in the synthesis of alkyne-linker-drugs constructs. Amide bond formation may be achieved from a carboxylic acid and a (primary or secondary) amine by one of two methods: (a) in situ activation of the carboxylic acid in the presence of the amine or (b) prior conversion of the carboxylic acid to an activated ester, optionally followed by purification, then reaction with the amine without requiring further activating agents. Two alternative routes towards an activated linker-drug featuring an amide bond are indicated in FIG. 3. Even though the route via the activated esters may involve an additional chemical conversion, the overall performance of the bioconjugate preparation method is improved.

The invention thus concerns a method for the preparation of an alkyne-linker-payload construct of structure Q-L-C(O)—NR³—D (1), comprising reacting (i) an alkyne compound of structure Q-L-C(O)-X (2), wherein
Q is an alkyne moiety selected from the group consisting of terminal alkyne and (hetero)cycloalkyne,
L is a linker, and
X is a leaving group selected from halogen, SR¹, 0-succinimidyl, O-(hetero)aryl(R²)₁₋₅, wherein R¹ is selected from C₁-C₆ alkyl and (hetero)aryl; and R² is C₁-C₆ alkyl, halogen or NO₂;
with (ii)a molecule of structure D-NHR³ (3), wherein
D is a payload, and
R³ is selected from hydrogen, optionally substituted C₁-C₂₄ alkyl, optionally substituted aryl.

The invention further concerns a method for preparing a bioconjugate, comprising:
(a) preparing a cycloalkyne-linker-payload construct of structure Q-L-C(O)-NR3-D (1) using the method according to any one of the preceding claims; and
(b) reacting the cycloalkyne-linker-payload construct with a biomolecule containing a moiety that is reactive towards an alkyne in a cycloaddition, to form a bioconjugate wherein the payload is covalently attached to the biomolecule.

The invention further concerns an alkyne compound of structure Q-L-C(O)-X (2), wherein:
Q is an alkyne moiety selected from the group consisting of terminal alkyne and (hetero)cycloalkyne,
L is a linker, and
X is a leaving group selected from halogen, SR¹, 0-succinimidyl, O-(hetero)aryl(R²)₁₋₅, wherein R¹ is selected from C₁-C₆ alkyl and (hetero)aryl; and R² is C₁-C₆ alkyl, halogen or NO₂.

### DESCRIPTION OF THE FIGURES

FIG. 1 shows cyclooalkynes suitable for metal-free click chemistry, including 7-membered, 8-membered, 9-membered and 10-membered rings. The list is not comprehensive, for example alkynes can be further activated by fluorination, by substitution of the aromatic rings or by introduction of heteroatoms in the aromatic ring.

FIG. 2 shows various exemplary 1,3-dipolar compounds or electron-poor heteroaromatic rings known to undergo cycloaddition with strained cycloalkynes.

FIG. 3 shows two alternative strategies for the preparation of a linker-drug, suitable for conjugation to a protein, based on amide bond-formation chemistry, involving either direct activation of carboxylic acid in presence of amine (route A) or a step-wise route involving prior conversion of carboxylic acid into activated ester (route B).

FIG. 4 shows the conversion of a BCN-linker-carboxylic acid (2) into a range of activated ester derivatives (3a-3f), based on para-nitrophenol (3a), pentafluorophenol (3b), pentachlorophenol (3c), 0-succinimide (3d), 1-hydroxybenzotriazole (3e) or 1-hydroxy-7-azabenzotriazole (3f).

FIG. 5 shows the acylation of a cytotoxic payload MMAF (4) with an activated ester derivative 3 to give BCN-linker-MMAF construct 5.

FIG. 6 shows a synthetic route for the preparation of cyclooctyne-modified PBD dimer 7 from Alloc-protected precursor 6, either by direct activation of carboxylic acid 2 or by acylation with an activated ester 3.

FIG. 7A-7D show stability plots for compounds 3a-3d.

FIG. 8 shows the stability plot for compound 2.

FIG. 9 shows the acylation of a cytotoxic payload va-PABC-PBD with an activated ester derivative 14 to give BCN-linker-PBD construct 16. This synthetic procedure is performed in Examples 11-17.

FIG. 10 shows the acylation of a cytotoxic payload va-PABC-MMAE with an activated ester derivative 20 to give BCN-linker-MMAE construct 21. This synthetic procedure is performed in Examples 18-22.

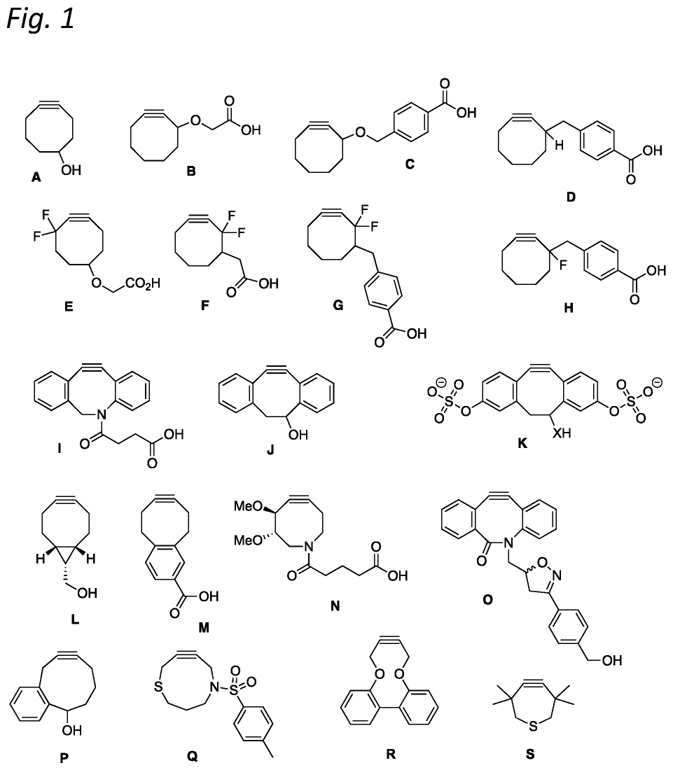

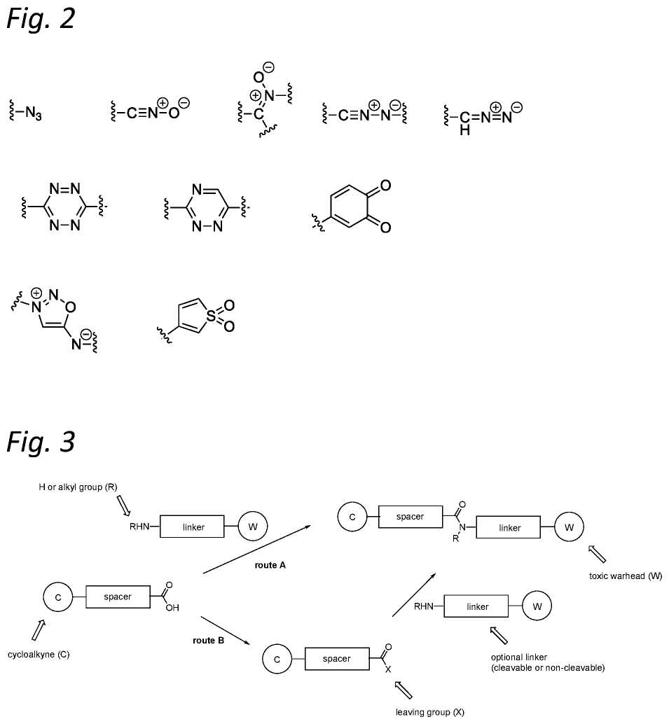

### DETAILED DESCRIPTION OF THE INVENTION

### Definitions

The verb “to comprise”, and its conjugations, as used in this description and in the claims is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

The compounds disclosed in this description and in the claims may comprise one or more asymmetric centres, and different diastereomers and/or enantiomers may exist of the compounds. The description of any compound in this description and in the claims is meant to include all diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the invention of the present application is not limited to that specific enantiomer.

The compounds may occur in different tautomeric forms. The compounds according to the invention are meant to include all tautomeric forms, unless stated otherwise. When the structure of a compound is depicted as a specific tautomer, it is to be understood that the invention of the present application is not limited to that specific tautomer.

The compounds disclosed in this description and in the claims may further exist as exo and endo diastereoisomers. Unless stated otherwise, the description of any compound in the description and in the claims is meant to include both the individual exo and the individual endo diastereoisomers of a compound, as well as mixtures thereof. When the structure of a compound is depicted as a specific endo or exo diastereomer, it is to be understood that the invention of the present application is not limited to that specific endo or exo diastereomer.

Furthermore, the compounds disclosed in this description and in the claims may exist as cis and trans isomers. Unless stated otherwise, the description of any compound in the description and in the claims is meant to include both the individual cis and the individual trans isomer of a compound, as well as mixtures thereof. As an example, when the structure of a compound is depicted as a cis isomer, it is to be understood that the corresponding trans isomer or mixtures of the cis and trans isomer are not excluded from the invention of the present application. When the structure of a compound is depicted as a specific cis or trans isomer, it is to be understood that the invention of the present application is not limited to that specific cis or trans isomer.

The compounds according to the invention may exist in salt form, which are also covered by the present invention. The salt is typically a pharmaceutically acceptable salt, containing a pharmaceutically acceptable anion. The term “salt thereof” means a compound formed when an acidic proton, typically a proton of an acid, is replaced by a cation, such as a metal cation or an organic cation and the like. Where applicable, the salt is a pharmaceutically acceptable salt, although this is not required for salts that are not intended for administration to a patient. For example, in a salt of a compound the compound may be protonated by an inorganic or organic acid to form a cation, with the conjugate base of the inorganic or organic acid as the anionic component of the salt.

The term “pharmaceutically accepted” salt means a salt that is acceptable for administration to a patient, such as a mammal (salts with counter ions having acceptable mammalian safety for a given dosage regime). Such salts may be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions known in the art and include, for example, sodium, potassium, calcium, magnesium, ammonium, tetraalkylammonium, etc., and when the molecule contains a basic functionality, salts of organic or inorganic acids, such as hydrochloride, hydrobromide, formate, tartrate, besylate, mesylate, acetate, maleate, oxalate, etc.

The term “protein” is herein used in its normal scientific meaning. Herein, polypeptides comprising about 10 or more amino acids are considered proteins. A protein may comprise natural, but also unnatural amino acids.

The term “antibody” is herein used in its normal scientific meaning. An antibody is a protein generated by the immune system that is capable of recognizing and binding to a specific antigen. An antibody is an example of a glycoprotein. The term antibody herein is used in its broadest sense and specifically includes monoclonal antibodies, polyclonal antibodies, dimers, multimers, multi-specific antibodies (e.g. bispecific antibodies), antibody fragments, and double and single chain antibodies. The term “antibody” is herein also meant to include human antibodies, humanized antibodies, chimeric antibodies and antibodies specifically binding cancer antigen. The term “antibody” is meant to include whole immunoglobulins, but also antigen-binding fragments of an antibody. Furthermore, the term includes genetically engineered antibodies and derivatives of an antibody. Antibodies, fragments of antibodies and genetically engineered antibodies may be obtained by methods that are known in the art. Typical examples of antibodies include, amongst others, abciximab, rituximab, basiliximab, palivizumab, infliximab, trastuzumab, efalizumab, alemtuzumab, adalimumab, tositumomab-1131, cetuximab, ibrituximab tiuxetan, omalizumab, bevacizumab, natalizumab, ranibizumab, panitumumab, eculizumab, certolizumab pegol, golimumab, canakinumab, catumaxomab, ustekinumab, tocilizumab, ofatumumab, denosumab, belimumab, ipilimumab and brentuximab.

An “antibody fragment” is herein defined as a portion of an intact antibody, comprising the antigen-binding or variable region thereof. Examples of antibody fragments include Fab, Fab′, F(ab′)₂, and Fv fragments, diabodies, minibodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragments of any of the above which immunospecifically bind to a target antigen (e.g., a cancer cell antigen, a viral antigen or a microbial antigen).

An “antigen” is herein defined as an entity to which an antibody specifically binds.

The terms “specific binding” and “specifically binds” is herein defined as the highly selective manner in which an antibody or antibody binds with its corresponding epitope of a target antigen and not with the multitude of other antigens. Typically, the antibody or antibody derivative binds with an affinity of at least about 1×10⁻⁷ M, and preferably 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “substantial” or “substantially” is herein defined as a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

A “linker” is herein defined as a moiety that connects two or more elements of a compound. For example in an antibody-conjugate, an antibody and a payload are covalently connected to each other via a linker. A linker may comprise one or more linkers and spacer-moieties that connect various moieties within the linker.

A “polar linker” is herein defined as a linker that contains structural elements with the specific aim to increase polarity of the linker, thereby improving aqueous solubility. A polar linker may for example comprise one or more units, or combinations thereof, selected from ethylene glycol, a carboxylic acid moiety, a sulfonate moiety, a sulfone moiety, an acylated sulfamide moiety, a phosphate moiety, a phosphinate moiety, an amino group or an ammonium group.

A “spacer” or spacer-moiety is herein defined as a moiety that spaces (i.e. provides distance between) and covalently links together two (or more) parts of a linker. The linker may be part of e.g. a linker-construct, the linker-conjugate or a bioconjugate, as defined below.

A “self-immolative group” is herein defined as a part of a linker in an antibody-drug conjugate with a function is to conditionally release free drug at the site targeted by the ligand unit. The activatable self-immolative moiety comprises an activatable group (AG) and a self-immolative spacer unit. Upon activation of the activatable group, for example by enzymatic conversion of an amide group to an amino group or by reduction of a disulfide to a free thiol group, a self-immolative reaction sequence is initiated that leads to release of free drug by one or more of various mechanisms, which may involve (temporary) 1,6-elimination of a p-aminobenzyl group to a p-quinone methide, optionally with release of carbon dioxide and/or followed by a second cyclization release mechanism. The self-immolative assembly unit can part of the chemical spacer connecting the antibody and the payload (via the functional group).

Alternatively, the self-immolative group is not an inherent part of the chemical spacer, but branches off from the chemical spacer connecting the antibody and the payload.

An “activatable group” is herein defined as a functional group attached to an aromatic group that can undergo a biochemical processing step such as proteolytic hydrolysis of an amide bond or reduction of a disulphide bond, upon which biochemical processing step a self-immolative process of the aromatic group will be initiated. The activatable group may also be referred to as “activating group”.

A “bioconjugate” is herein defined as a compound wherein a biomolecule is covalently connected to a payload via a linker. A bioconjugate comprises one or more biomolecules and/or one or more payloads. Antibody-conjugates, such as antibody-payload conjugates and antibody-drug-conjugates are bioconjugates wherein the biomolecule is an antibody.

A “biomolecule” is herein defined as any molecule that can be isolated from nature or any molecule composed of smaller molecular building blocks that are the constituents of macromolecular structures derived from nature, in particular nucleic acids, proteins, glycans and lipids. Examples of a biomolecule include an enzyme, a (non-catalytic) protein, a polypeptide, a peptide, an amino acid, an oligonucleotide, a monosaccharide, an oligosaccharide, a polysaccharide, a glycan, a lipid and a hormone.

The term “payload” refers to the moiety that is covalently attached to a targeting moiety such as an antibody, but also to the molecule that is released from the conjugate upon uptake of the protein conjugate and/or cleavage of the linker. Payload thus refers to the monovalent moiety having one open end which is covalently attached to the targeting moiety via a linker, which is in the context of the present invention referred to as D, and also to the molecule that is released therefrom.

The invention

The present inventors have found that activated ester derivatives of alkyne compounds unexpectedly facilitate the covalent attachment of the alkyne compound to an amine-functionalized payload molecule. The activated ester derivatives can be conveniently prepared by activation of precursor carboxylic acid and are (a) highly stable and (b) provide for smooth and high-yielding attachment to a cytotoxic payload, with or without a cleavable linker. As such, the activated ester compounds of the present invention are ideal intermediates in the preparation of bioconjugates, wherein a biomolecule is covalently attached to a cytotoxic payload. The activated ester derivative is first attached to the payload, and subsequently the alkyne moiety is reacted with an appropriately functionalized biomolecule.

The present invention thus in a first aspect provides a method for the preparation of an alkyne-linker-payload construct of structure Q-L-C(O)—NR³—D (1), comprising reacting (i) an alkyne compound of structure Q-L-C(O)-X (2) with (ii) a molecule of structure D-NHR³ (3). In a second aspect, the invention provides a method for preparing a bioconjugate, comprising (a) preparing a cycloalkyne-linker-payload construct of structure Q-L-C(O)—NR³—D (1) using the method according to the first aspect, and (b) reacting the cycloalkyne-linker-payload construct with a biomolecule containing a moiety that is reactive towards an alkyne in a cycloaddition, to form a bioconjugate wherein the payload is covalently attached to the biomolecule. In a third aspect, the invention concerns the alkyne compound of structure Q-L-C(O)-X (2). Related to the use according to the second aspect, the invention also concerns the use of the alkyne compound according to the third aspect for preparing an alkyne-linker-payload construct of structure Q-L-C(O)—NR³—D (1), or the use of the alkyne compound according to the third aspect as intermediate in the synthesis of a bioconjugate.

As will be clear from the context of the present invention, everything defined herein for the method according to the first aspect equally applies to the first step of the method according to the second aspect. Likewise, the structural definition of the compound according to the third aspect equally applies to the respective structural elements in the methods according to the first and second aspects, and vice versa.

The alkyne compound of structure Q-L-C(O)-X (2)

The alkyne compound according to the present invention has the structure Q-L-C(O)-X (2), wherein:
Q is an alkyne moiety selected from the group of terminal alkyne and (hetero)cycloalkyne,
L is a linker, and
X is a leaving group selected from halogen, SR¹, O-succinimidyl, O-(hetero)aryl(R²)₁₋₅, wherein R¹ is selected from C₁-C₆ alkyl and (hetero)aryl; and R² is C₁-C alkyl, halogen or NO₂

The alkyne compound according to the invention is a useful intermediate in the synthesis of bioconjugates. More specifically, it can be used for preparing an alkyne-linker-payload construct of structure Q-L-C(O)—NR³—D (1). The alkyne compound according to the invention, bearing an activated ester, or more stable than the corresponding carboxylic acids and also provide a higher yield in the preparation of the alkyne-linker-payload construct.

Alkyne moiety Q

Alkyne moiety Q is used in the final stage of the conjugation process to connect the linker-payload construct to the biomolecule and is thus conveniently inert in the reaction with the molecule of structure D-NHR³ (3). The alkynyl group may be a terminal alkyne or a cycloalkyne, including heterocycloalkynes.

In one embodiment, the alkyne moiety is a terminal alkyne, preferably the alkynyl group is according to structure (Q1) as shown below, wherein I is an integer in the range of 0 to 10, preferably in the range of 0 to 6. More preferably, I is 0, 1, 2, 3 or 4, more preferably I is 0, 1 or 2 and most preferably I is 0 or 1.

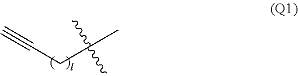

In an especially preferred embodiment, the alkynyl group is a (hetero)cycloalkynyl group, i.e. a heterocycloalkynyl group or a cycloalkynyl group, wherein the (hetereo)cycloalkynyl group is optionally substituted. Preferably, the (hetero)cycloalkynyl group is a (hetero)cycloheptynyl group, a (hetero)cyclooctynyl group, a (hetero)cyclononynyl group or a (hetero)cyclodecynyl group. Most preferably, the (hetero)cycloalkynyl group is a (hetero)cyclooctynyl group, wherein the (hetero)cyclooctynyl group is optionally substituted. Herein, the alkynes and (hetero)cycloakynes may optionally be substituted. Preferably, Q comprises a (hetero)cyclooctyne moiety according to structure (Q2) below. In a further preferred embodiment, the (hetero)cyclooctynyl group is according to structure (Q36), (Q37) or (Q38) as defined further below. Preferred examples of the (hetero)cyclooctynyl group include structure (Q3), also referred to as a DIBO group, (Q4), also referred to as a DIBAC group, or (Q5), also referred to as a BARAC group, (Q6), also referred to as a COMBO group, and (Q7), also referred to as a BCN group, all as shown below, wherein Y¹ is O or NR¹¹, wherein R¹¹ is independently selected from the group consisting of hydrogen, a linear or branched C₁-C₁₂ alkyl group or a C₄-C₁₂ (hetero)aryl group. The aromatic rings in (Q3) are optionally O-sulfonylated at one or more positions, whereas the rings of (Q4) and (Q5) may be halogenated at one or more positions. A particularly preferred cycloalkynyl group is a bicyclo[6.1.0]non-4-yn-9-yl] group (BCN group), which is optionally substituted. Preferably, the bicyclo[6.1.0]non-4-yn-9-yl] group is according to formula (Q7) as shown below, wherein V is (CH₂), and I is an integer in the range of 0 to 10, preferably in the range of 0 to 6. More preferably, I is 0, 1, 2, 3 or 4, more preferably I is 0, 1 or 2 and most preferably I is 0 or 1. In the context of group (Q7), I is most preferably 1.

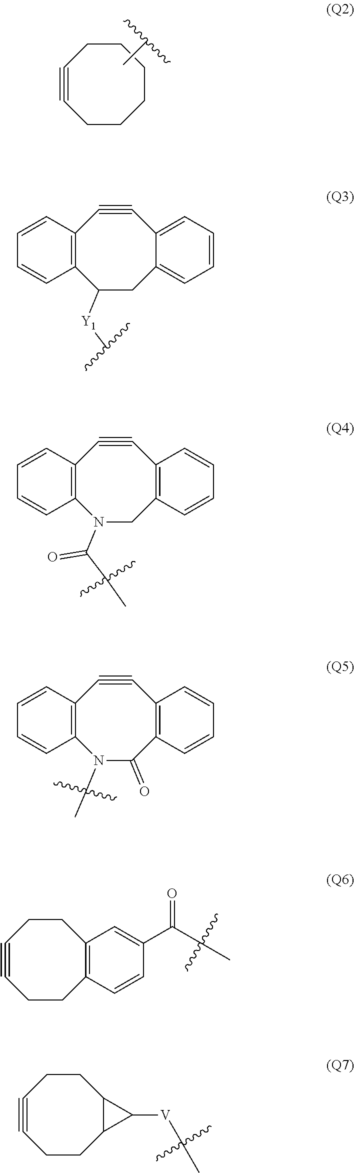

In a further preferred embodiment, the alkynyl group is selected from the group consisting of (Q8)-(Q21) depicted here below.

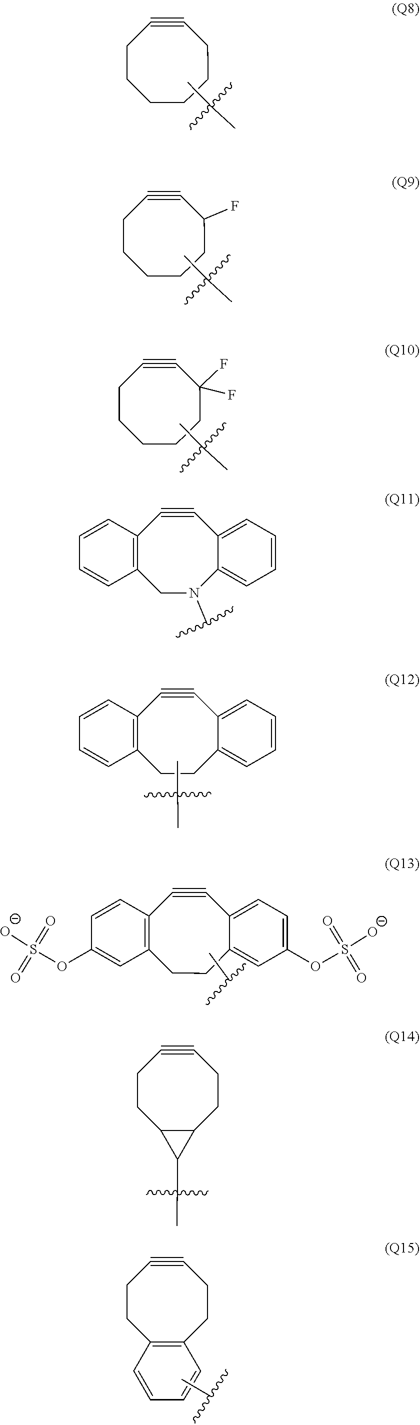

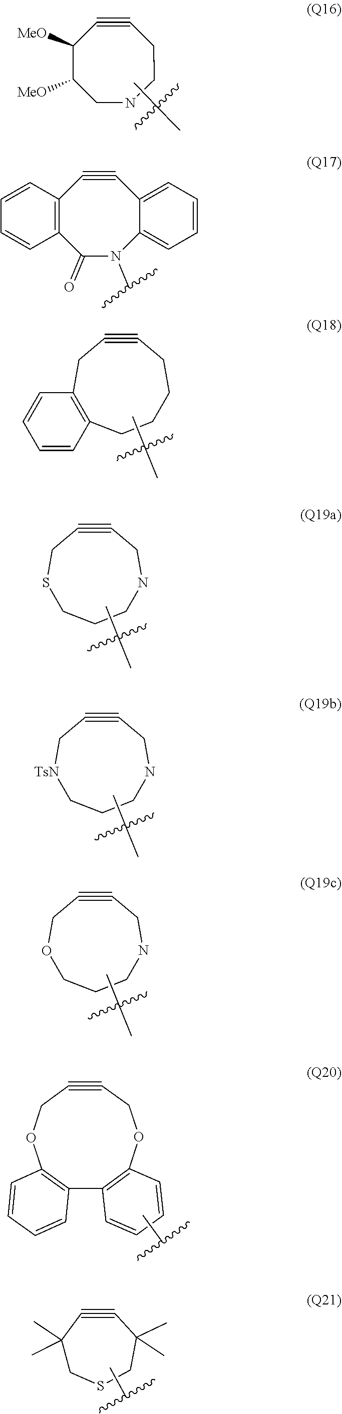

Herein, the connection to L, depicted with the wavy bond, may be to any available carbon or nitrogen atom of Q.

In a further preferred embodiment, the alkynyl group is selected from the group consisting of (Q22)-(Q35c) depicted here below.

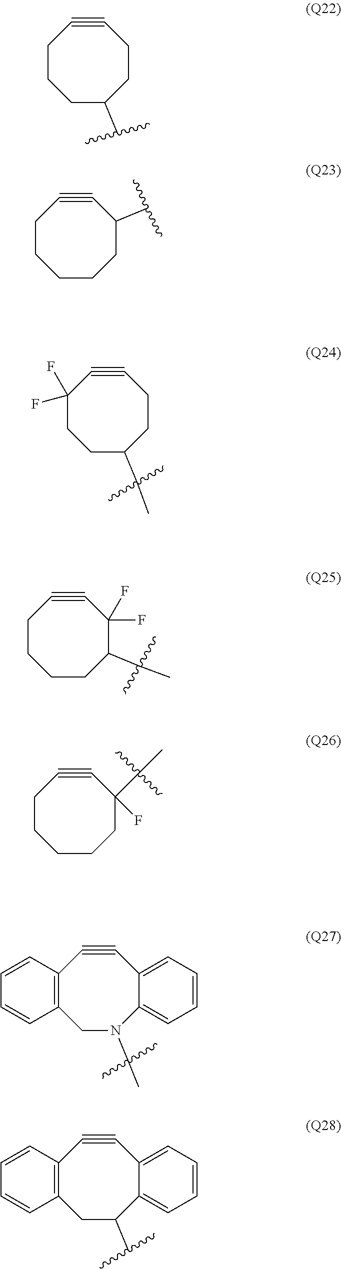

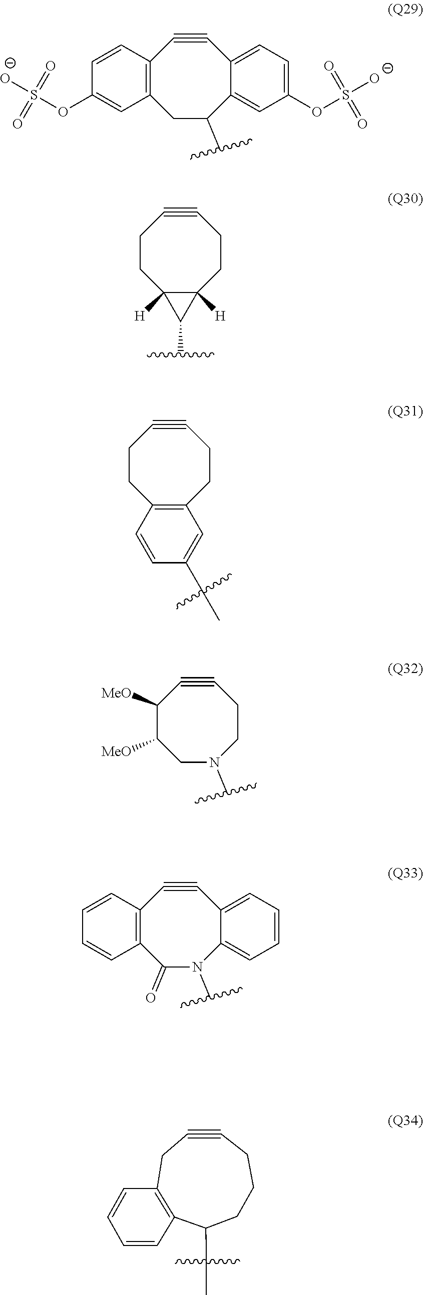 [SMILES: *C1Cc2cc(OS(=O)(=O)[O-])ccc2C#Cc2ccc(OS(=O)(=O)[O-])cc21 ; *[C@H]1[C@H]2CCC#CCC[C@@H]12 ; *c1ccc2c(c1)CCC#CCC2 ; *N1CCC#C[C@H](OC)[C@@H](OC)C1 ; *N1C(=O)c2ccccc2C#Cc2ccccc21 ; *C1CCCC#CCc2ccccc21]

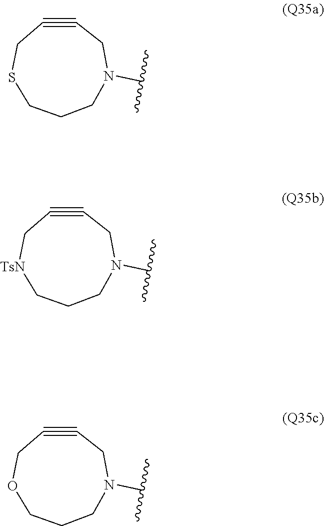 [SMILES: *N1CC#CCSCCC1 ; *N1CC#CCN(S(=O)(=O)c2ccc(C)cc2)CCC1 ; *N1CC#CCOCCC1]

In an especially preferred embodiment, reactive group Q comprises an (hetero)cycloalkynyl group and is according to structure (Q36):

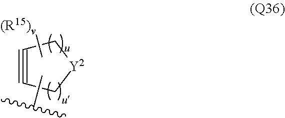

Herein:
R¹⁵ is independently selected from the group consisting of hydrogen, halogen, —OR¹⁶, —NO₂, —CN, —S(O)₂R¹⁶, —S(O)₃( ), C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S or NR³¹, wherein each R³¹ is individually is R¹⁵ or -(L¹)ₙ-(L²)ₒ-(L³)ₚ-(L⁴)_{q}-D;
u is 0, 1, 2, 3, 4 or 5;
u′ is 0, 1, 2, 3, 4 or 5, wherein u+u′=4, 5, 6, 7 or 8;
v=an integer in the range 8-16.

In a preferred embodiment, u+u′=4, 5 or 6, more preferably u+u′=5. Typically, v=(u+u′)×2 or [(u+u′)×2]-1. In a preferred embodiment, v=8, 9 or 10, more preferably v=9 or 10, most preferably v=10.

In an especially preferred embodiment, reactive group Q comprises an alkynyl group and is according to structure (Q37):

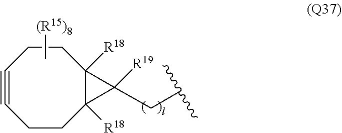

Herein:
R¹⁵ is independently selected from the group consisting of hydrogen, halogen, —OR¹⁶, —NO₂, —CN, —S(O)₂R¹⁶, —S(O)₃( ),C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, -(L¹)ₙ-(L²)ₒ-(L³)ₚ-(L⁴)_{q}-halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted; and
I is an integer in the range 0 to 10.

In a preferred embodiment of the reactive group according to structure (Q37), R¹⁵ is independently selected from the group consisting of hydrogen, halogen, —OR¹⁶, C₁-C alkyl groups, C₅-C₆ (hetero)aryl groups, wherein R¹⁶ is hydrogen or C₁-C₆ alkyl, more preferably R¹⁵ is independently selected from the group consisting of hydrogen and C₁-C alkyl, most preferably all R¹⁵ are H. In a preferred embodiment of the reactive group according to structure (Q37), R¹⁸ is independently selected from the group consisting of hydrogen, C₁-C alkyl groups, most preferably both R¹⁸ are H. In a preferred embodiment of the reactive group according to structure (Q37), R¹⁹ is H. In a preferred embodiment of the reactive group according to structure (Q37), I is 0 or 1, more preferably I is 1. An especially preferred embodiment of the reactive group according to structure (Q37) is the reactive group according to structure (Q20).

In an especially preferred embodiment, reactive group Q comprises an alkynyl group and is according to structure (Q38):

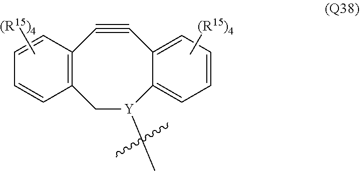

Herein:
R¹⁵ is independently selected from the group consisting of hydrogen, halogen, —OR¹⁶, —NO₂, —CN, —S(O)₂R¹⁶, —S(O)₃( ), C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

In a preferred embodiment of the reactive group according to structure (Q38), R¹⁵ is independently selected from the group consisting of hydrogen, halogen, —OR¹⁶, —S(O)₃( ), C₁-C alkyl groups, C₅-C₆ (hetero)aryl groups, wherein R¹⁶ is hydrogen or C₁-C₆ alkyl, more preferably R¹⁵ is independently selected from the group consisting of hydrogen and —S(O)₃( ). In a preferred embodiment of the reactive group according to structure (Q38), Y is N or CH, more preferably Y═N.

### Linker L

Linkers, also referred to as linking units, are well known in the art and any suitable linker may be used. In the final alkyne-linker-payload construct, the payload is chemically connected to a terminal alkyne or a cyclic alkyne via a cleavable or non-cleavable linker. The linker may contain one or more branch-points for attachment of multiple payloads to a single terminal alkyne or cyclic alkyne. Preparation of the alkyne-linker-drug can be achieved by chemical methods described herein.

The linker may for example be selected from the group consisting of linear or branched C₁-C₂₀₀ alkylene groups, C₂-C₂₀0 alkenylene groups, C₂-C₂₀0 alkynylene groups, C₃-C₂₀₀ cycloalkylene groups, C₅-C₂₀₀ cycloalkenylene groups, C₈-C₂₀₀ cycloalkynylene groups, C₇-C₂₀₀ alkylarylene groups, C₇-C₂₀0 arylalkylene groups, C₈-C₂₀0 arylalkenylene groups, C₉-C₂₀₀ arylalkynylene groups. Optionally the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups may be substituted, and optionally said groups may be interrupted by one or more heteroatoms, preferably 1 to 100 heteroatoms, said heteroatoms preferably being selected from the group consisting of O, S(O)y and NR¹², wherein y is 0, 1 or 2, preferably y=2, and R¹² is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups. The linker may contain (poly)ethylene glycoldiamines (e.g. 1,8-diamino-3,6-dioxaoctane or equivalents comprising longer ethylene glycol chains), (poly)ethylene glycol or (poly)ethylene oxide chains, (poly)propylene glycol or (poly)propylene oxide chains and 1,z-diaminoalkanes wherein z is the number of carbon atoms in the alkane, and may for example range from 2-25.

In a preferred embodiment, linker L comprises a sulfamide group, preferably a sulfamide group according to structure (L1):

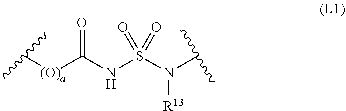

The wavy lines represent the connection to the remainder of the compound, typically to Q and to C(O)X, optionally via a spacer. Preferably, the (O)ₐC(O) moiety is connected to Q and the NR¹³ moiety to C(O)X.

In structure (L1), a=0 or 1, preferably a=1, and R¹³ is selected from the group consisting of hydrogen, C₁-C₂₄ alkyl groups, C₃-C₂₄ cycloalkyl groups, C₂-C₂₄ (hetero)aryl groups, 03
C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is a second occurrence of C(O)X connected to N via a spacer moiety, preferably Sp² as defined here below.

In a preferred embodiment, R¹³ is hydrogen or a C₁-C₂₀ alkyl group, more preferably R¹³ is hydrogen or a C₁-C₁s alkyl group, even more preferably R¹³ is hydrogen or a C₁-C₁₀ alkyl group, wherein the alkyl group is optionally substituted and optionally interrupted by one or more heteroatoms selected from O, S and NR¹⁴, preferably O, wherein R¹⁴ is independently selected from the group consisting of hydrogen and C₁-C₄ alkyl groups. In a preferred embodiment, R¹³ is hydrogen. In another preferred embodiment, R¹³ is a C₁-C₂₀ alkyl group, more preferably a C₁-C₁s alkyl group, even more preferably a C₁-C₁₀ alkyl group, wherein the alkyl group is optionally interrupted by one or more O-atoms, and wherein the alkyl group is optionally substituted with an —OH group, preferably a terminal —OH group. In this embodiment it is further preferred that R¹³ is a (poly)ethylene glycol chain comprising a terminal —OH group. In another preferred embodiment, R¹³ is selected from the group consisting of hydrogen, methyl, ethyl, n-propyl, i-propyl, n-butyl, s-butyl and t-butyl, more preferably from the group consisting of hydrogen, methyl, ethyl, n-propyl and i-propyl, and even more preferably from the group consisting of hydrogen, methyl and ethyl. Yet even more preferably, R¹³ is hydrogen or methyl, and most preferably R¹³ is hydrogen.

In a preferred embodiment, the linker is according to structure (L2):

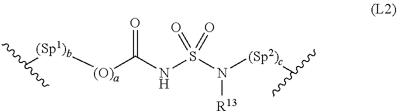

Herein, a, R¹³ and the wavy lines are as defined above, Sp¹ and Sp² are independently spacer moieties and b and c are independently 0 or 1. Preferably, b=0 or 1 and c=1, more preferably b=0 and c=1. In one embodiment, spacers Sp¹ and Sp² are independently selected from the group consisting of linear or branched C₁-C₂₀0 alkylene groups, C₂-C₂₀0 alkenylene groups, C₂-C₂₀0 alkynylene groups, C₃-C₂₀₀ cycloalkylene groups, C₅-C₂₀₀ cycloalkenylene groups, C₈-C₂₀₀ cycloalkynylene groups, C₇-C₂₀0 alkylarylene groups, C₇-C₂₀0 arylalkylene groups, C₈-C₂₀0 arylalkenylene groups and C-C₂₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. When the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are interrupted by one or more heteroatoms as defined above, it is preferred that said groups are interrupted by one or more O-atoms, and/or by one or more S—S groups.

More preferably, spacer moieties Sp¹ and Sp², if present, are independently selected from the group consisting of linear or branched C₁-C₁₀₀ alkylene groups, C₂-C₁₀₀ alkenylene groups, C₂-C₁₀₀ alkynylene groups, C₃-C₁₀₀ cycloalkylene groups, C₅-C₁₀₀ cycloalkenylene groups, C₈-C₁₀₀ cycloalkynylene groups, C₇-C₁₀₀ alkylarylene groups, C₇-C₁₀₀ arylalkylene groups, C₈-C₁₀₀ arylalkenylene groups and C₉-C₁₀₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Even more preferably, spacer moieties Sp¹ and Sp², if present, are independently selected from the group consisting of linear or branched C₁-C₀ alkylene groups, C₂-C₅₀ alkenylene groups, C₂-C₀ alkynylene groups, C₃-C₅₀ cycloalkylene groups, C₅-C₅₀ cycloalkenylene groups, C₈-C₅₀ cycloalkynylene groups, C₇-C₀ alkylarylene groups, C₇-C₅₀ arylalkylene groups, C₈-C₀ arylalkenylene groups and C₉-C₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

Yet even more preferably, spacer moieties Sp¹ and Sp², if present, are independently selected from the group consisting of linear or branched C₁-C₂₀ alkylene groups, C₂-C₂₀ alkenylene groups, C₂-C₂₀ alkynylene groups, C₃-C₂₀ cycloalkylene groups, C₅-C₂₀ cycloalkenylene groups, C₈-C₂₀ cycloalkynylene groups, C₇-C₂₀ alkylarylene groups, C₇-C₂₀ arylalkylene groups, C₈-C₂₀ arylalkenylene groups and C₉-C₂₀ arylalkynylene groups, the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted.

In these preferred embodiments it is further preferred that the alkylene groups, alkenylene groups, alkynylene groups, cycloalkylene groups, cycloalkenylene groups, cycloalkynylene groups, alkylarylene groups, arylalkylene groups, arylalkenylene groups and arylalkynylene groups are unsubstituted and optionally interrupted by one or more heteroatoms selected from the group of O, S and NR²⁰, preferably O, wherein R²⁰ is independently selected from the group consisting of hydrogen and C₁-C₄ alkyl groups, preferably hydrogen or methyl.

Most preferably, spacer moieties Sp¹ and Sp², if present, are independently selected from the group consisting of linear or branched C₁-C₂₀ alkylene groups, the alkylene groups being optionally substituted and optionally interrupted by one or more heteroatoms selected from the group of O, S and NR²⁰, wherein R²⁰ is independently selected from the group consisting of hydrogen, C₁-C₂₄ alkyl groups, C₂-C₂₄ alkenyl groups, C₂-C₂₄ alkynyl groups and C₃-C₂₄ cycloalkyl groups, the alkyl groups, alkenyl groups, alkynyl groups and cycloalkyl groups being optionally substituted. In this embodiment, it is further preferred that the alkylene groups are unsubstituted and optionally interrupted by one or more heteroatoms selected from the group of O, S and NR²⁰, preferably O and/or S-S, wherein R²⁰ is independently selected from the group consisting of hydrogen and C₁-C₄ alkyl groups, preferably hydrogen or methyl.

Another class of suitable linkers comprises cleavable linkers. Cleavable linkers are well known in the art. For example Shabat et al., Soft Matter2012, 6, 1073, incorporated by reference herein, discloses cleavable linkers comprising self-immolative moieties that are released upon a biological trigger, e.g. an enzymatic cleavage or an oxidation event. Some examples of suitable cleavable linkers are peptide-linkers that are cleaved upon specific recognition by a protease, e.g. cathepsin, plasmin or metalloproteases, or glycoside-based linkers that are cleaved upon specific recognition by a glycosidase, e.g. glucoronidase, or nitroaromatics that are reduced in oxygen-poor, hypoxic areas.

Linker L may further contain a peptide spacer as known in the art, preferably a dipeptide or tripeptide spacer as known in the art, preferably a dipeptide spacer. Although any dipeptide or tripeptide spacer may be used, preferably the peptide spacer is selected from Val-Cit, Val-Ala, Val-Lys, Val-Arg, AcLys-Val-Cit, AcLys-Val-Ala, Phe-Cit, Phe-Ala, Phe-Lys, Phe-Arg, Ala-Lys, Leu-Cit, Ile-Cit, Trp-Cit, Ala-Ala-Asn, Ala-Asn, more preferably Val-Cit, Val-Ala, Val-Lys, Phe-Cit, Phe-Ala, Phe-Lys, Ala-Ala-Asn, more preferably Val-Cit, Val-Ala, Ala-Ala-Asn. In one embodiment, the peptide spacer is Val-Cit. In one embodiment, the peptide spacer is Val-Ala.

The peptide spacer may also be attached to the payload, wherein the amino end of the peptide spacer is conveniently used as amine group in the method according to the first aspect of the invention.

In a preferred embodiment, the peptide spacer is represented by general structure (L3):

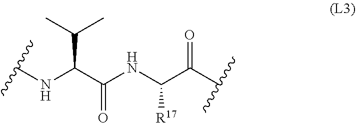

Herein, R¹⁷=CH₃ (Val) or CH₂CH₂CH₂NHC(O)NH₂ (Cit). The wavy lines indicate the connection to the remainder of the molecule, preferably the peptide spacer according to structure (L3) is connected to Q via NH and to C(O)X via C(O).

Linker L may further contain a self-cleavable spacer, also referred to as self-immolative spacer. The self-cleavable spacer may also be attached to the payload. Preferably, the self-cleavable spacer is para-aminobenzyloxycarbonyl (PABC) derivative, more preferably a PABC derivative according to structure (L4).

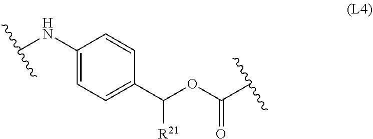

Herein, the wavy lines indicate the connection to the remainder of the molecule.

Typically, the PABC derivative is connected via NH to Q, typically via a spacer, and via OC(O) to C(O)X, typically via a spacer.

R²¹ is H, R²² or C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃
C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups. Preferably, R²² is C₃-C₁₀ (hetero)cycloalkyl or polyalkylene glycol. The polyalkylene glycol is preferably a polyethylene glycol or a polypropylene glycol, more preferably —(CH₂CH₂O)ₛH or —(CH₂CH₂CH₂O)ₛH. The polyalkylene glycol is most preferably a polyethylene glycol, preferably —(CH₂CH₂O)ₛH, wherein s is an integer in the range 1-10, preferably 1-5, most preferably s=1, 2, 3 or 4. More preferably, R²¹ is H or C(O)R²², wherein R²²=4-methyl-piperazine or morpholine. Most preferably, R²¹ is H.

The activated ester

Linker L connects alkyne Q with the activated ester C(O)X. Activated esters are known in the art, and can readily be prepared from a carboxylic acid precursor C(O)OH.

X is a leaving group selected from halogen, SR¹, 0-succinimidyl, O-(hetero)aryl(R²)₁₋₅, wherein R¹ is selected from C₁-C₆ alkyl and (hetero)aryl; and R² is C₁-C alkyl, halogen or NO₂. In one embodiment, X=halogen, preferably X=Cl or F, more preferably X=C₁. In another embodiment, X=SR¹, wherein R¹ is selected from C₁-C₆ alkyl and (hetero)aryl, preferably R¹=ethyl or pentafluorophenyl. In another embodiment, X=0-succinimidyl. In another embodiment, X=O-(hetero)aryl(R²)₁₋₅, wherein R² is C₁-C₆ alkyl, halogen or NO₂, preferably R²=Cl, F or NO₂, more preferably X=O-Ph(R²)₄₋₅, wherein R₂=Cl or F, O-Ph(R²), wherein R₂=NO₂, or benzotriazole. In an especially preferred embodiment, X is selected from halogen, 0-succinimide (X1), O-benzotriazole (X2), 0-pyridinotriazole (X3), 4-nitrophenol (X4), 2,3,5,6-tetrafluorophenol (X5), 2,3,4,5,6-pentafluorophenol (X6) or 2,3,4,5,6-pentachlorophenol (X7).

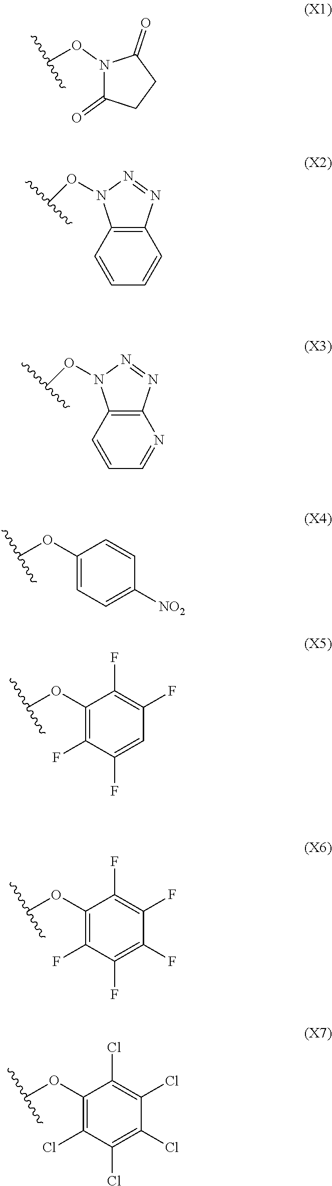

The alkyne compound of structure Q-L-C(O)-X (2) is preferably represented by a structure selected from the group consisting of (2a)-(2p):

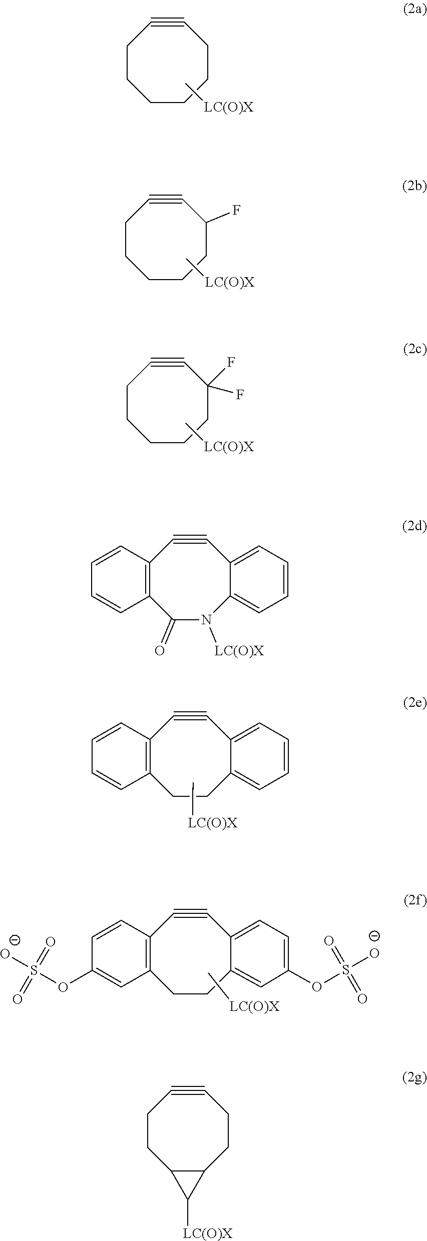

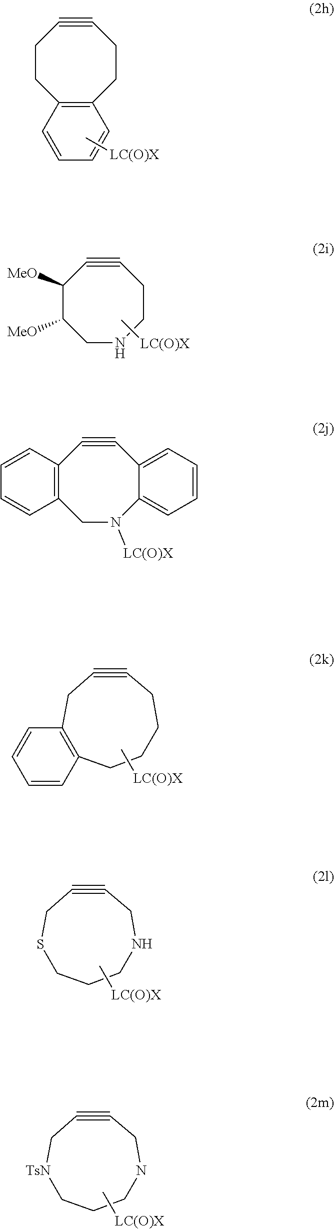

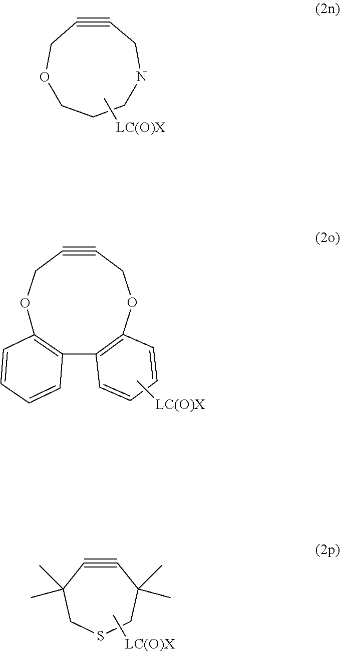

In a further preferred embodiment, the alkyne compound of structure Q-L-C(O)-X (2) is represented by a structure selected from the group consisting of (2q)-(2ad):

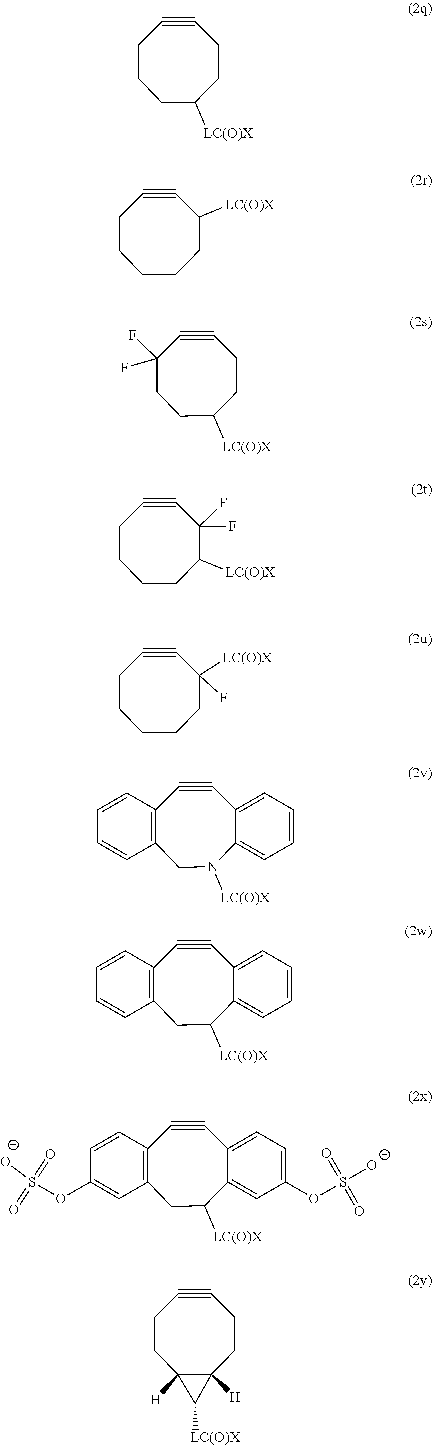

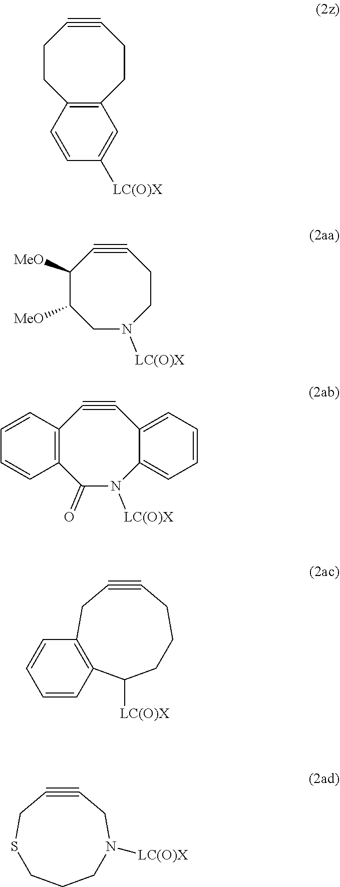

### The Method for Preparing an Alkyne-Linker-Payload Construct of Structure Q-L-C(O)—NR³—D (1)

In the method according to the first aspect, the alkyne compound of structure Q-L-C(O)-X (2), as defined above, is reacted with a molecule of structure D-NHR³ (3). Herein:
D is a payload, and
R³ is selected from hydrogen, optionally substituted C₁-C₂₄ alkyl, optionally substituted aryl.

During the reaction, the activated ester is coupled to the amine-functionalized payload D, to form a covalent connection between alkyne Q and payload D. Such activated ester couplings are known in the art, and can be performed in any way possible. The skilled person knows how to perform the reaction according to the present aspect, by bringing both molecules into contact. Either during the in situ activation procedure or during the reaction of activated ester with amine, additional reagents (base, catalysts) may be present in the reaction mixture.

Reagents known in the art for in situ activation of carboxylic acid include, but are not limited to, carbodiimides (e.g. DCC, DIC, EDC), uronium agents (e.g. HBTU, HATU), phosphonium agents (e.g. BOP, pyBOP, pyAOP) and others (e.g. T3P, DEPBT).

The reaction according to the present aspect of the invention proceeds smoothly with primary (R³=H) and secondary amines (R³=optionally substituted C₁-C₂₄ alkyl, optionally substituted aryl). In a preferred embodiment, R³=hydrogen or C₁-C alkyl. More preferably, R³=hydrogen.

In one embodiment, the alkyne compound of structure (2) is prepared by activation of Q-L-C(O)OH. Such activation of carboxylic acids is well-known in the art. Thus, the method according to this aspect may further comprise the step of activating a carboxylic acid compound of structure Q-L-C(O)OH to form the alkyne compound of structure Q-L-C(O)-X (2), which is then subjected to the present reaction.

D is a payload. Payload molecules are well-known in the art, especially in the field of antibody-drug conjugates, as the moiety that is covalently attached to the antibody and that is released therefrom upon uptake of the conjugate and/or cleavage of the linker. Payloads containing a primary or secondary amine functionality are well-known in the art. The skilled person is aware of such payload, and knows how to introduce a primary or secondary amine functionality if needed. This can for example be accomplished by reacting a hydroxyl moiety with 6-aminohexanoic acid (Ahx) to introduce an ester-linked primary amino moiety. Thus, the Ahx moiety will be part of the payload in the context of the present invention.

In a preferred embodiment, the payload D is a cytotoxin, preferably selected from the group consisting of taxanes, anthracyclines, camptothecins, epothilones, mytomycins, combretastatins, vinca alkaloids, maytansinoids, enediynes, duocarmycins, tubulysins, amatoxins, auristatins, pyrrolobenzodiazepines (or dimers thereof), indolino-benzodiazepines (or dimers thereof), isoquinolino-benzodiazepines (or dimers thereof), amanitins, ligands for radioisotopes, therapeutic peptides (or fragments thereof), kinase inhibitors, MEK inhibitors, KSP inhibitors, Bcl inhibitors, NAMPT inhibitors, PARP inhibitors and analogues or combinations or prodrugs thereof. In a preferred embodiment, D is a maytansinoid, pyrrolobenzodiazepine (PBD) or the dimer thereof, more preferably MMAE or pyrrolobenzodiazepine dimer. In an especially preferred embodiment, D is pyrrolobenzodiazepine or the dimer thereof, most preferably pyrrolobenzodiazepine dimer.

### The Method for Preparing a Bioconjugate

The invention further concerns a method for preparing a bioconjugate, wherein the method according to the first aspect is step (a). More specifically, the method according to the second aspect comprises:
(a) preparing a cycloalkyne-linker-payload construct of structure Q-L-C(O)—NR³—D (1) using the method according to the first aspect; and
(b) reacting the cycloalkyne-linker-payload construct with a biomolecule containing a moiety that is reactive towards an alkyne in a cycloaddition, to form a bioconjugate wherein the payload is covalently attached to the biomolecule.

The preparation of step (a) is extensively described above, which equally applies to the present aspect. The reaction of step (b) is a conjugation wherein a biomolecule is covalently attached to a payload to form a bioconjugate. Herein the alkyne forms a covalent attachment to the reactive moiety on the biomolecule. Such conjugation reactions with alkyne moieties are known in the art, e.g. from WO 2014/065661, and may typically take the form of a 1,3-dipolar cycloaddition or a [4+2] cycloaddition.

The biomolecule is thus functionalized with a reactive group that is reactive towards an alkyne in a cycloaddition, or in other words that is capable of forming a covalent attachment with an alkyne moiety. The skilled person is aware of such reactive groups, which may be selected from azide, tetrazine, triazine, nitrone, nitrile oxide, nitrile imine, diazo compound, ortho-quinone, dioxothiophene and sydnone. Preferred structures for the reactive group are structures (P1)-(P9) depicted here below.

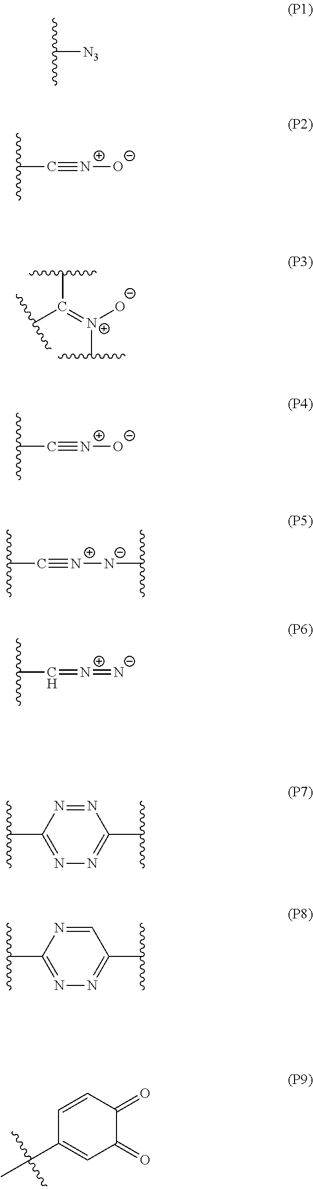

Herein, the wavy bond represents the connection to the biomolecule. For (P3), (P5), (P7) and (P8), the biomolecule can be connected to any one of the wavy bonds. Preferably, the reactive group is selected from azides or tetrazines. Most preferably, the reactive group is an azide.

The nature of the biomolecule is mot limited in the context of the present reaction. In a preferred embodiment, the biomolecule is selected from the group consisting of proteins (including glycoproteins and antibodies), polypeptides, peptides, glycans, lipids, nucleic acids, oligonucleotides, polysaccharides, oligosaccharides, enzymes, hormones, amino acids and monosaccharides. More preferably from the group consisting of proteins, polypeptides, peptides and glycans. Especially preferred are proteins as biomolecules.

### EXAMPLES

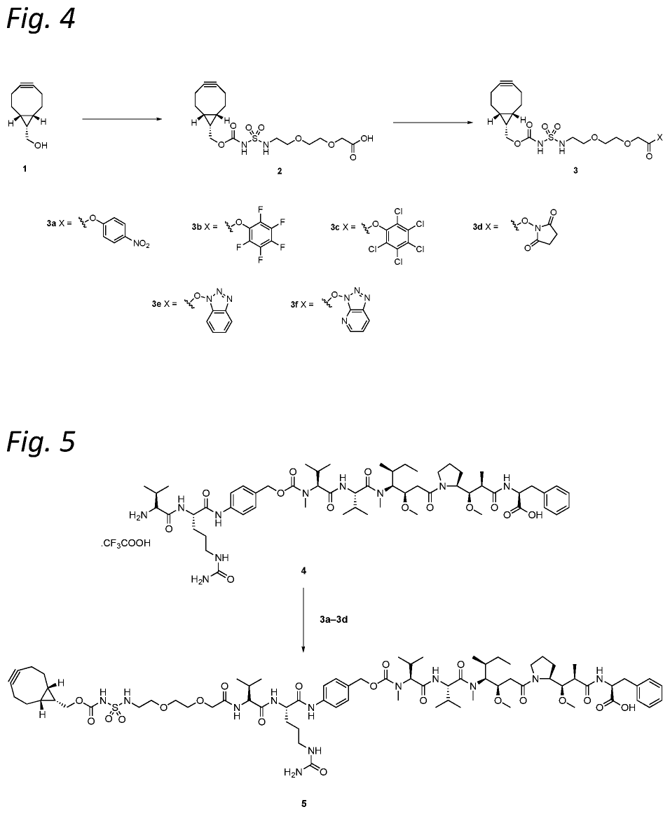

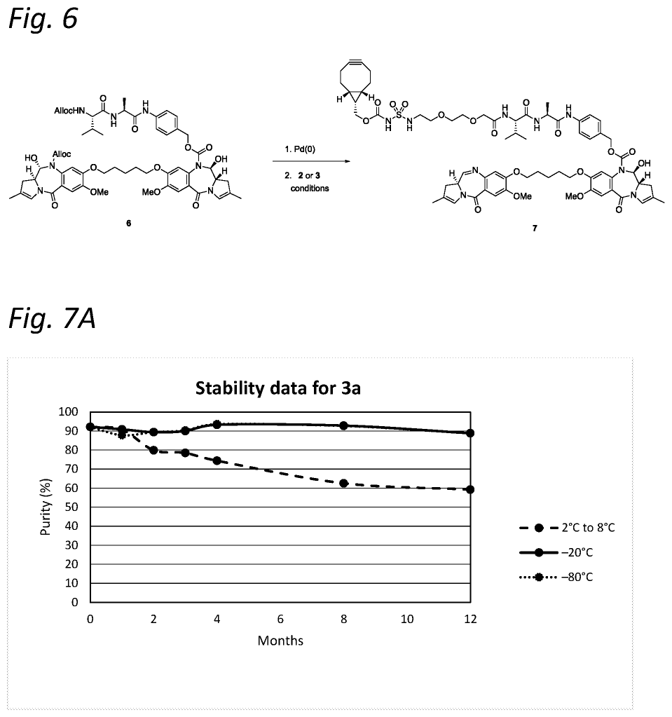

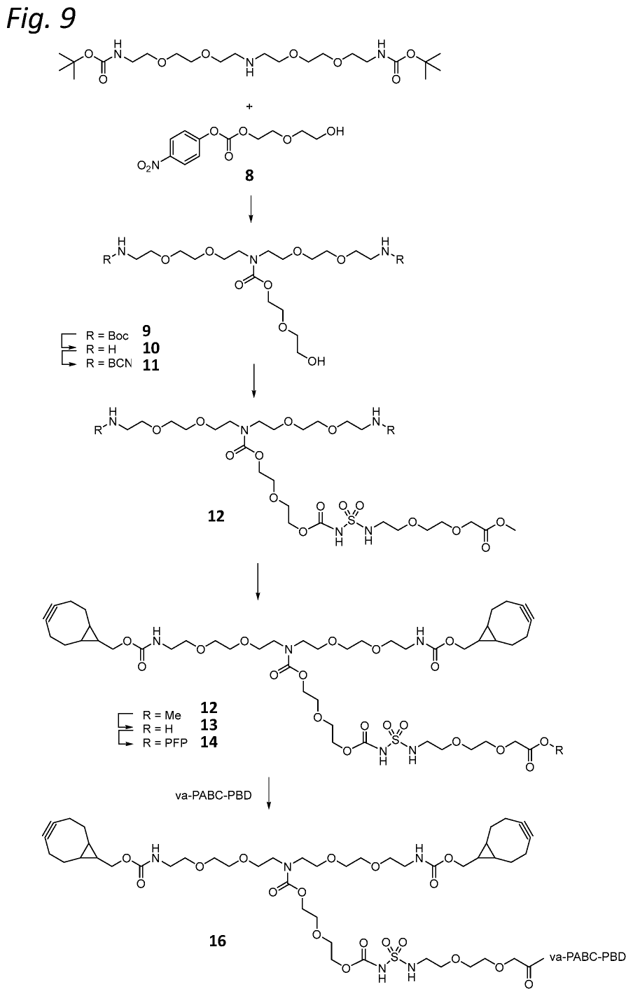

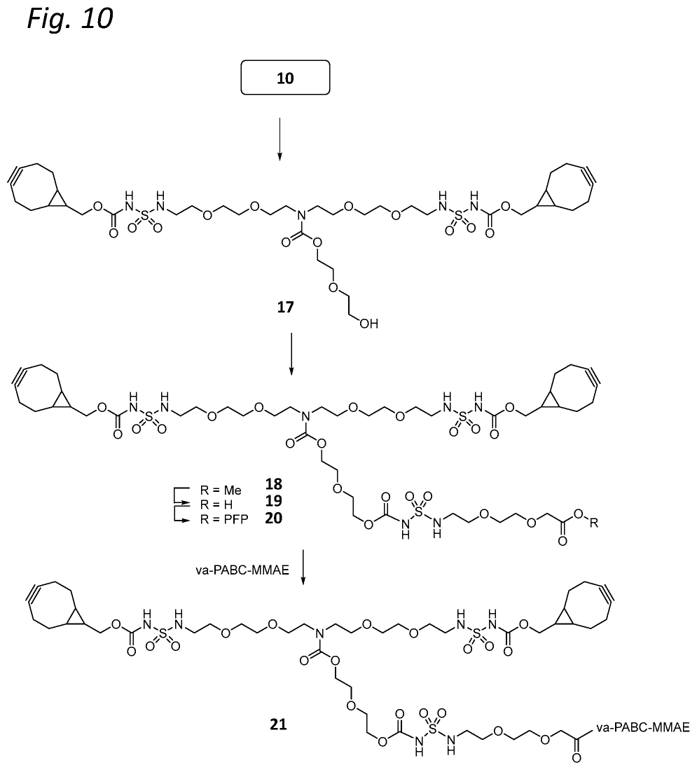

Synthetic preparation of a linker-drug with a cyclooctyne reactive moiety (BCN) and cytoxic payload of Examples 1-6 is highlighted in FIGS. 4-6. The synthetic procedure of Examples 11-17 is depicted in FIG. 9. The synthetic procedure of Examples 18-22 is depicted in FIG. 10.

### Example 1. Synthesis of Compound 2

Compound 2 can be prepared as described in WO2018146189 (see intermediate 3).

A solution of BCN alcohol 1 (0.384 g, 2.55 mmole) in MeCN (25 mL) under a N₂ atmosphere was cooled to 0° C., and chlorosulfonyl isocyanate (CSI) was added was added dropwise (0.255 mL, 415 mg, 2.93 mmole, 1.15 equiv.). After stirring for 15 minutes, Et₃N was added dropwise (1.42 mL, 1.03 g, 10.2 mmole, 4 equiv.) and stirring was continued for another 10 minutes. Next, a solution of 2-(2-(2-aminoethoxy)ethoxy)acetic acid (1.0 g, 6.1 mmole, 2.4 equiv.) in H₂O (5 mL) was added and the reaction mixture was stirred to room temperature for 2 h. After this time, CHCl₃ (50 mL) and H₂O (100 mL) were added, and the layers were separated. To the aqueous layer in a separatory funnel was added CH₂Cl₂ (100 mL) and the pH was adjusted to 4 with 1 N HCl, before separation of layers. The water layer was extracted twice with CH₂Cl₂ (2×100 mL), the organic layers were combined and dried (Na₂SO₄), filtered and concentrated. The residue was purified by flask column chromatography on silica, elution with CH₂Cl₂ to 20% MeOH in CH₂Cl₂. Yield 0.42 g (1.0 mmole, 39%) of 2 as a colorless sticky wax.

Various activated ester-derivatives of BCN-linker-drug are indicated in FIG. 4.

### Example 2. Preparation of Activated Ester 3a

To a solution 2 (673 mg, 1.61 mmol) in DCM (100 mL) were added N-ethyl-N′-(3-dimethylaminopropyl)carbodiimide hydrochloride (370 mg, 1.93 mmol) and p-nitrophenol (268 mg, 1.93 mmol). The resulting mixture was stirred overnight and concentrated. The crude product was purified by silica gel chromatography (30%→70% EtOAc in heptane). The desired product was obtained as a colorless wax (202 mg, 0.374 mmol, 23%). ¹H NMR (400 MHz, CDC3) 5 (ppm) 8.32-8.25 (m, 2H), 7.39-7.32 (m, 2H), 4.46 (s, 2H), 4.26 (d, J=8.5 Hz, 2H), 3.85-3.80 (m, 2H), 3.74-3.70 (m, 2H), 3.69-3.65 (m, 2H), 3.32 (t, J=4.9 Hz, 2H), 2.35-2.15 (m, 6H), 1.61-1.45 (m, 2H), 1.37 (quintet, J=8.7 Hz, 1H), 1.03-0.91 (m, 2H).

### Example 3. Preparation of Activated Ester 3b

To a solution of 2 (1.10 g, 2.62 mmol) in DCM (100 mL) were added N-ethyl-N′-(3-dimethylaminopropyl)carbodiimide hydrochloride (603 mg, 3.15 mmol) and pentafluorophenol (580 mg, 3.15 mmol). The resulting mixture was stirred for 30 min and a sample of 20 mL was concentrated. The crude product was purified by silica gel chromatography (20%→70% EtOAc in heptane). The remainder was stirred for 21 h, concentrated and the crude product was purified by silica gel chromatography (20%→70% EtOAc in heptane). The desired products of both columns were combined and obtained as a colorless thick oil (776 mg, 1.33 mmol, 51%). ¹H NMR (400 MHz, CDCl3) δ (ppm) 5.74 (bs, 1H), 4.54 (s, 2H), 4.26 (d, J=8.2 Hz, 2H), 3.83-3.78 (m, 2H), 3.73-3.68 (m, 2H), 3.67-3.62 (m, 2H), 3.35-3.27 (m, 2H), 2.35-2.14 (m, 6H), 1.62-1.46 (m, 2H), 1.38 (quintet, J=8.7 Hz, 1H), 1.03-0.91 (m, 2H).

### Example 4. Preparation of Activated Ester 3c

To a solution of 2 (906 mg, 2.17 mmol) in DCM (100 mL) were added N-ethyl-N′-(3-dimethylaminopropyl)carbodiimide hydrochloride (0.50 g, 2.60 mmol) and pentachlorophenol (0.69 g, 2.60 mmol). The resulting mixture was stirred for 18 h, concentrated and the residue was purified by silica gel chromatography (20%→70% EtOAc in heptane). The desired products was obtained as a white solid (858 mg, 1.29 mmol, 59%). ¹H NMR (400 MHz, CDCl3) δ (ppm) 5.76-5.64 (m, 1H), 4.56 (s, 2H), 4.27 (d, J=8.2 Hz, 2H), 3.86-3.80 (m, 2H), 3.74-3.69 (m, 2H), 3.69-3.64 (m, 2H), 3.36-3.29 (m, 2H), 2.35-2.16 (m, 6H), 1.61-1.47 (m, 2H), 1.38 (quintet, J=8.8 Hz, 1H), 1.03-0.92 (m, 2H).

### Example 5. Preparation of Activated Ester 3d

To a solution of 2 (799 mg, 1.91 mmol) in DCM (100 mL) were added N-ethyl-N′-(3-dimethylaminopropyl)carbodiimide hydrochloride (0.44 g, 2.31 mmol) and N-hydroxysuccinimide (266 mg, 2.31 mmol). The resulting mixture was stirred for 17 h, concentrated and the residue was purified by silica gel chromatography (50%→100% EtOAc in heptane). The desired products was obtained as a slightly yellow oil (723 mg, 1.40 mmol, 73%). ¹H NMR (400 MHz, CDCl3) δ (ppm) 5.77-5.66 (m, 1H), 4.52 (s, 2H), 4.27 (d, J=8.2 Hz, 2H), 3.83-3.78 (m, 2H), 3.71-3.67 (m, 2H), 3.66-3.61 (m, 2H), 3.37-3.28 (m, 2H), 2.87 (s, 4H), 2.36-2.16 (m, 6H), 1.62-1.48 (m, 2H), 1.39 (quintet, J=9.0 Hz, 1H), 1.04-0.94 (m, 2H).

### Example 6A. Preparation of BCN-Linker-MMAF 5 by Acylation of 4 with Activated Ester 3a

To a solution of 1.0 mg of 4 (100 μL) was added a 10% (v/v) solution of Et₃N in DMF (3.3 μL) and a solution of 3a (0.43 mg, 0.80 μmol) in DMF (27 μL). The reaction mixture was left overnight. UPLC-MS analysis showed full conversion to the desired product. LCMS (ESI+) calculated for C₇₅H₁₁₇N₁₂O₂₀S⁺ (M+H⁺) 1537.82 found 1537.72.

### Example 6B. Preparation of BCN-Linker-MMAF 5 by Acylation of 4 with Activated Ester 3b

To a solution of 1.0 mg of 4 (100 μL) was added a 10% (v/v) solution of Et₃N in DMF (3.3 μL) and a solution of 3b (0.47 mg, 0.80 μmol) in DMF (27 μL). The reaction mixture was left overnight. UPLC-MS analysis showed full conversion to the desired product. LCMS (ESI+) calculated for C₇₅H₁₁₇N₁₂O₂₀S⁺ (M+H⁺) 1537.82 found 1537.72.

### Example 6C. Preparation of BCN-Linker-MMAF 5 by Acylation of 4 with Activated Ester 3c

To a solution of 1.0 mg of 4 (100 μL) was added a 10% (v/v) solution of Et₃N in DMF (3.3 μL) and a solution of 3c (0.53 mg, 0.80 μmol) in DMF (27 μL). The reaction mixture was left overnight. UPLC-MS analysis showed full conversion to the desired product. LCMS (ESI+) calculated for C₇₅H₁₁₇N₁₂O₂₀S⁺(M+H⁺) 1537.82 found 1537.72.

### Example 6D. Preparation of BCN-Linker-MMAF 5 by Acylation of 4 with Activated Ester 3d

To a solution of 1.0 mg of 4 (100 μL) was added a 10% (v/v) solution of Et₃N in DMF (3.3 μL) and a solution of 3d (0.43 mg, 0.80 μmol) in DMF (27 μL). The reaction mixture was left overnight. UPLC-MS analysis showed full conversion to the desired product. LCMS (ESI+) calculated for C₇₅H₁₁₇N₁₂O₂₀S⁺(M+H⁺) 1537.82 found 1537.72.

### Example 7. Alloc Deprotection of Compound 4

The deprotection can be performed as described in WO2018146189 or by this procedure.

A stream of nitrogen gas was passed through a solution of 6 (20 mg, 18 μmol) in DCM (5.0 mL). A solution of pyrrolidine (3.2 mg, 3.8 μL, 45 μmol) in DCM (107 μL) and a solution of Pd(PPh₃)₄(3.1 mg, 2.7 μmol) in DCM (86 μL) were added. A stream of nitrogen gas was passed through the resulting reaction mixture. After 10 min, the reaction mixture was diluted with DCM (20 mL) and washed with saturated aqueous NH₄Cl (20 mL). After separation, the aqueous phase was extracted with DCM (2×20 mL). The combined organic layers were dried (Na₂SO₄) and concentrated. The residue was dissolved in a mixture of DMF (400 μL) and MeCN (400 μL) and filtered. The filter was rinsed with DMF (100 μL) and the combined filtrates were purified by RP-HPLC (C18, 5%→90% MeCN (0.1% formic acid) in water (0.1% formic acid). The fractions containing the desired product were filtered over a PL-HCO3 MP SPE column and concentrated.

The residue was taken up in MeCN (25 mL) and the resulting mixture was concentrated (2x), which yielded the desired product as a white film (13.5 mg, 14.6 μmol, 81%). LCMS (ESI+) calculated for C₄₉H₆₀N₇O₁₁⁺ (M+H⁺) 922.43 found 922.72.

### Example 8. Direct Activation of Carboxylic Acid 2

The direct activation can be performed as described in WO2018146189 or by this procedure.

To a solution of the deprotected PBD dimer in DCM (5 mL) is added a solution of 2 (15 mg, 36 μmol) in DCM (0.8 mL). The resulting mixture is added to solid EDC.HCl (4.7 mg, 25 μmol), DCM (5 mL) was added and the mixture stirred. After 30 minutes, DCM (30 mL) is added and the resulting mixture is washed with water (30 mL). After separation, the aqueous phase is extracted with DCM (30 mL). The combined organic layers are dried (Na₂SO₄) and concentrated.

The residue is purified by RP-HPLC (30-90% MeCN (no acid) in H₂O with 0.01% formic acid).

The HPLC collection tubes are filled with 5% aqueous (NH₄)HCO₃ before collection. The combined HPLC fractions are extracted with DCM (3×20 mL). The combined organic layers are dried (Na₂SO₄) and concentrated. The product 7 is obtained as slightly yellow/white oil (21 mg, 16 μmol, 67% over two steps). The conversion of the reaction can be monitored through LCMS analysis. Column: XBridge BEH C₁₈ Intelligent Speed (IS) Column, 130A, 3.5 pri (4.6 mm×20 mm). Mobile phase A: Water (0.1% formic acid), Mobile phase B (0.1% formic acid). Detection with PDA and ESI+. LCMS (ESI+) calculated for C₆₆H₈₄N₉O₁₈S⁺(M+H⁺) 1322.56 found 1322.84.

### Example 9. Acylation of deprotected PBD dimer with activated ester 3b

To a solution of the deprotected PBD dimer (13.5 mg, 14.6 μmol) in DCM (5 mL) was added a solution of 3b (15.8 mg, 27 μmol) in DCM (0.62 μL) and Et₃N (5.5 mg, 7.5 μL). The resulting reaction mixture was stirred for 19 h and purified via silica gel column chromatography (0%→10% MeOH in DCM). The desired product was obtained as a white film in quantitative yield (19.5 mg, 14.7 μmol). LCMS (ESI+) calculated for C₆₆H₈₄N₉O₁₈S⁺(M+H⁺) 1322.56 found 1322.84.

### Example 10. Stability studies

For compound 3a, 3b, 3c, 3d or 2, aliquoted samples were stored at indicated temperatures (2-8° C. and −20° C. and −80° C.). Before analysis, samples were allowed to reach room temperature. HPLC samples were prepared: 0.33 mg/mL MeCN for 3a; 0.66 mg/mL MeCN for 3b and 3c; 7.5 mg/mL DMF/MeCN (1:4). RP-UPLC-MS analysis of the samples was performed on a Waters Acquity UPLC, equipped with PDA and MS-detector and a ACQUITY UPLC BEH C₁₈ Column, 130 Å, 1.7 μm, 2.1 mm×150 mm, Waters P/N 186002353. Buffer A: 0.1% formic acid in acetonitrile. Buffer B: 0.1% formic acid in water, as follows:

| Time (minutes) | Buffer A (%) | Buffer B (%) |
|---|---|---|
| 0.00 | 5 | 95 |
| 0.38 | 5 | 95 |
| 7.50 | 95 | 5 |
| 8.25 | 95 | 5 |
| 8.44 | 5 | 95 |
| 11.25 | 5 | 95 |

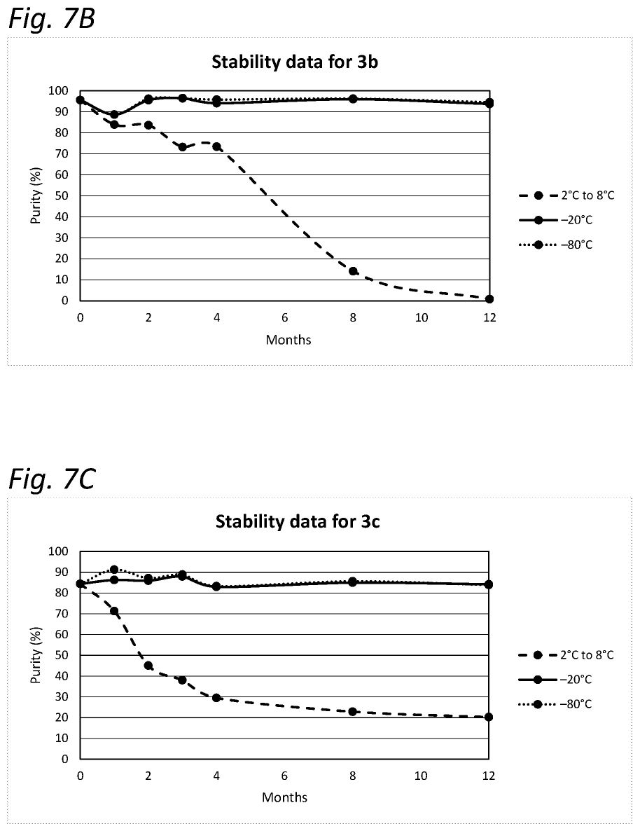

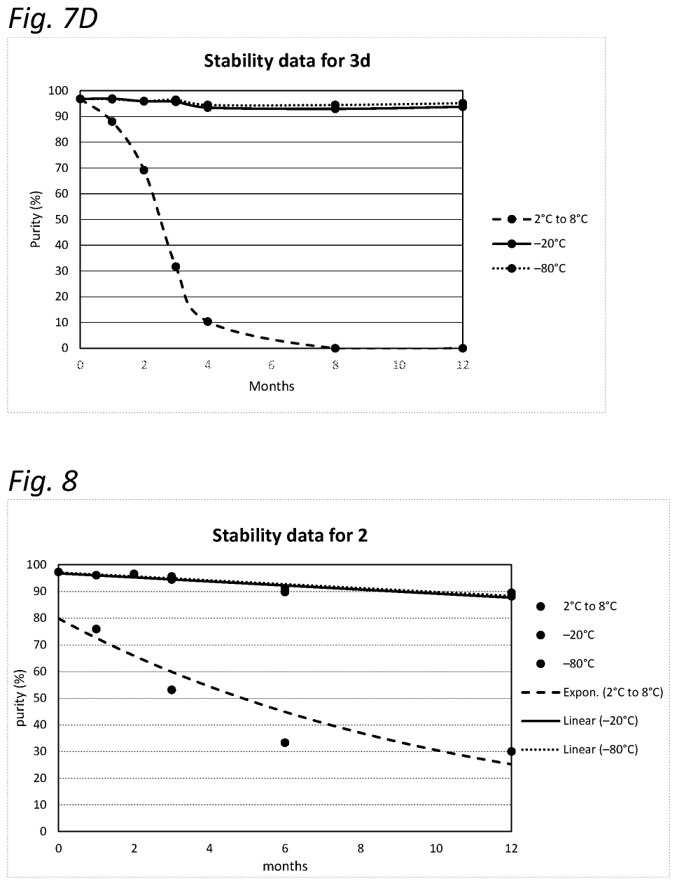

Purity was determined via integration of UV-peaks at 215 nM. The stability plots are provided in FIGS. 7A-7D and 8.

### Example 11. Syntheses of Mono-Alcohol 9

To a solution of NH-(PEG₂-Boc)₂ (1.00 g, 2.08 mmol, 1 equiv.) in 2 mL CH₂Cl₂ was added 8 (848 mg, 3.13 mmol, 1.5 equiv.) dissolved in 1 mL CH₂Cl₂ followed by the addition of Et₃N (872 μL, 6.25 mmol, 3 equiv.) and 1-hydroxybenzotriazole hydrate (319 mg, 2.08 mmol, 1 equiv.).

The almost clear yellow solution was stirred for 5 days at room temperature. UPLC-MS showed complete consumption of compound 8. TLC (EtOAc/heptane 1:1 and 3:1) confirmed complete consumption of compound 8. The reaction mixture was concentrated in vacuo and the residue was taken up in 5 mL MeCN followed by the addition of 6 mL aqueous NaOH (2N, 6 equiv.).

Stirring was continued at room temperature for 2 hours after which the mixture was diluted with 8 mL H₂O and 8 mL MeCN providing a homogeneous mixture. Stirring was continued at room temperature for another 2.5 hours. UPLC-MS showed complete conversion to the desired product. The mixture was extracted with CH₂Cl₂ (3×50 mL) and the combined organic layers were concentrated in vacuo. The obtained oil was purified by A.F.C.C. (Sepacore); 25 G Premium; flow 20 ml/min; 10 ml per fraction; runtime 22.50 minutes; 0%→12% CH₃OH/CH₂Cl₂. Product containing fractions were combined and concentrated in vacuo to yield compound 9 as a pale yellow oil (1.14 g, 1.86 mmol; 86%). ¹H NMR (400 MHz, CDCl₃) δ (ppm) 4.28-4.23 (m, 2H), 3.77-3.68 (m, 4H), 3.66-3.56 (m, 14H), 3.56-3.48 (m, 8H), 3.35-3.26 (m, 4H), 1.44 (s, 18H). UPLC-MS (ESI+) calculated for C₄₇H₇₈N₇O₂₃S₃+(M+H)⁺ 1204.43 found 1204.62.

### Example 12. Boc Deprotection Producing Compound 10

Compound 9 (1.14 g, 95% Wt, 1.77 mmol, 1 equiv.) was dissolved in CH₂Cl₂ (2 mL) followed by addition of 4M HCl in dioxane (2.35 mL, 9.40 mmol). The reaction mixture was stirred for 16h at room temperature after which UPLC analysis still showed the presence of intermediates and starting material. Therefore additional 4M HCl in dioxane (2.35 mL, 9.40 mmol) was added and stirring at room temperature was continued for another 4 hours. Now UPLC analysis showed near complete conversion. The reaction mixture was concentrated in vacuo and 2x co-evaporated with 4 mL n-heptane to yield compound 10 (858 mg, 1.77 mmol, quant.) as red/brown oil. This product was used without any further purification in the next step. ¹H NMR (400 MHz, DMSO-ds) 5 (ppm) 8.18-7.93 (br. s, 2H), 4.15-4.08 (m, 1H), 3.71-3.43 (m, 28H), 3.43-3.36 (m, 2H), 3.02-2.91 (m, 2H). UPLC-MS (ESI+) calculated for C₁₇H₃₈N₃O₈+(M+H)⁺ 412.26 found 412.57.

### Example 13. Acylation of Compound 10 with BCN-OSu Producing Compound 11

BCN-OSu (495 mg, 1.70 mmol, 2 equiv.) was dissolved in CH₂Cl₂ (4 mL) and added dropwise (in 6 minutes) to a solution of compound 10 (350 mg, 0.85 mmol, 1 equiv.) in 5.9 mL CH₂Cl₂ already containing 5 equiv. Et₃N. Extra Et₃N (237 μL, 1.70 mmol, 2 equiv.) was added and the resulting pale yellow solution was stirred at room temperature for 45 minutes. TLC (EtOAc/heptane 1:1) showed complete consumption of BCN-OSu. UPLC confirmed the presence of primarily product but also still some mono acylated product. Therefore additional BCN-OSu (50 mg, 0.17 mmol, 0.2 equiv) was added. After stirring the reaction for another hour at room temperature TLC analysis showed complete disappearance of compound 10. The reaction mixture was concentrated in vacuo and the residue was taken up in 4 mL CH₂Cl₂. The resulting suspension was diluted further with CH₂Cl₂ and purified by A.F.C.C. (Sepacore); 25 G HP; flow 20 ml/min; runtime 15 minutes; 0%→5% CH₃OH/CH₂Cl₂; 10 ml per fraction. Fractions containing product were concentrated in vacuo to yield 709.5 mg (0.93 mmol; 109%) of pale yellow oil which required another round of purification Hence the product was taken up in 5 mL CH₂Cl₂ containing 100 μL DMF to allow dissolution of all the material. The complete mixture was injected onto the column: A.F.C.C. (Sepacore); 12 G HP; flow 20 ml/min; runtime 15 minutes; 5%→10% CH₃OH in EtOAc followed by 15 minutes 0%→5% CH₃OH in CH₂Cl₂ and 7.5 minutes 5%→20% CH₃OH in CH₂Cl₂; 10 ml per fraction. Fractions containing product were concentrated in vacuo to yield 426.2 mg (0.56 mmol; 66%) as pale yellow oil. The product was still not pure and thus subjected to a third round of purification by A.F.C.C. (Sepacore); 12 G HP; flow 20 mL/min; 10 mL/fr; runtime 22.50 minutes; 0%→10% CH₃OH in EtOAc; Fractions containing product were concentrated in vacuo to yield pure compound 11 as colorless oil (289.8 mg, 0.38 mmol; 45%). ¹H NMR (400 MHz, CDCl₃) δ (ppm) 5.56-5.45 (br. s, 1H), 5.39-5.29 (br. s, 1H), 4.29-4.23 (m, 2H), 4.22-4.10 (m, 4H), 3.77-3.67 (m, 4H), 3.67-3.57 (m, 14H), 3.57-3.48 (m, 8H), 3.43-3.31 (m, 4H), 2.36-2.16 (m, 12H), 1.69-1.50 (m, 4H), 1.36 (quintet, J=8.7 Hz, 2H), 1.01-0.88 (m, 4H).

### Example 14. CSI Reaction of Compound 11 Producing Compound 12

Compound 11 (289.8 mg, 379.4 μmol, 1 equiv.) was dissolved in 5 mL dry CH₂Cl₂. Next chlorosulfonyl isocyanate (32.94 μL, 379.4 μmol, 1 equiv.) was added and the mixture was stirred for 5 minutes in which its color slowly changed to pale yellow. TLC (5% CH₃OH/CH₂Cl₂) still showed quite a lot of starting material present. Therefore another 0.2 equiv. of CSI (6.6 μL; 75.9 μmol) was added. Stirring was continued for 2 minutes and TLC analysis (5% CH₃OH/CH₂Cl₂) now showed an almost complete conversion. Next Et₃N (159 μL, 1.14 mmol, 3 equiv.) was added to the pale yellow reaction mixture. The resulting yellow clear reaction mixture was stirred for 17 minutes after which a solution of methyl 2-(2-(2-aminoethoxy)ethoxy)acetate,HCl (105.4 mg, 493.2 μmol, 1.3 equiv.) in 1 mL CH₂Cl₂ containing Et₃N (131 μL; 0.94 mmol, 2 equiv.) was added. The resulting reaction mixture was stirred at room temperature for 18 hours after which UPLC-MS and TLC analyses (10% CH₃OH in CH₂Cl₂) showed product formation. The reaction mixture was concentrated in vacuo and the yellow turbid residue was purified by A.F.C.C. (Sepacore); 12 G HP; flow 20 ml/min; runtime 22.50 minutes; 0%→5% CH₃OH/CH₂Cl₂; 10 ml per fraction; Fractions containing product were pooled and concentrated in vacuo to yield compound 12 as a colorless film (144.0 mg, 138 μmol; 36%). ¹H NMR (400 MHz, CDCl₃) δ (ppm) 6.05-5.78 (br. s, 1H), 5.42-5.28 (br. s, 2H), 4.34-4.27 (m, 2H), 4.27-4.21 (m, 2H), 4.21-4.10 (m, 6H), 3.77 (s, 3H), 3.75-3.58 (m, 22H), 3.58-3.47 (m, 8H), 3.42-3.33 (m, 4H), 3.33-3.25 (m, 2H), 2.36-2.16 (m, 12H), 1.70-1.50 (m, 4H), 1.36 (quintet, J=8.7 Hz, 2H), 1.01-0.88 (m, 4H). UPLC-MS (ESI+) calculated for C₄₇H₇₆N₅O₁₉S⁺(M+H)⁺ 1046.48 found 1046.77.

### Example 15. Methyl Ester Hydrolysis Producing Compound 13

Compound 12 (140 mg, 134 μmol, 1 equiv.) was dissolved in a 1:1 mixture of THF (1.5 mL) and H₂O (1.5 mL) resulting in a turbid solution. Next 5 equiv. of aqueous NaOH (1M, 669 μmol) were added and the clear yellow reaction mixture was stirred for 4.5 hours. TLC (1% AcOH in EtOAc) showed complete conversion to the desired product. The reaction mixture was poured into 15 mL EtOAc and 15 mL H₂O. The pH of the H₂O layer was adjusted to 3.5-4 with 1M HCl (650 μL). After shaking and separation of the layers (pH of the H₂O layer was still 3.5-4) the aqueous layer was extracted once more with 6 mL EtOAc. The organic layers were combined followed by drying over Na₂SO₄, filtration and concentrated in vacuo. The residue was co-evaporated once with 3 mL MeCN to yield compound 13 as pale yellow film (107.8 mg, 104.4 μmol; 78%). The product was used without further purification in the next step. UPLC-MS (ESI+) calculated for C₄₆H₇₄N₅O₁₉S⁺(M+H)⁺ 1032.47 found 1032.78.

### Example 16. PFP Ester Synthesis Producing Compound 14

A stock solution of 56.5 mg/mL pentafluorophenol in dry CH₂Cl₂ was prepared. Next, compound 15 (103 mg, 99.8 μmol, 1 equiv.) was dissolved in dry CH₂Cl₂ (6 mL) and EDC.HCl (25 mg, 0.13 mmol, 1.3 equiv.) was added followed by pentafluorophenol (22.8 mg, 124 μmol, 1.24 equiv.) and the reaction mixture was stirred for 15 hours. UPLC-MS showed clear product formation and almost no PFP was present anymore. The reaction mixture was concentrated in vacuo. The residue was taken up in dry CH₂Cl₂ and purified by A.F.C.C. (Sepacore); 4 G HP; flow 15 ml/min; 5 ml per fraction; runtime 15 minutes; 0%→40% acetone in CH₂Cl₂; Fractions containing product were pooled and concentrated in vacuo to yield compound 14 as a colorless oil (76.9 mg, 64.2 μmol; 64%). ¹H NMR (400 MHz, CDCl₃) δ (ppm) 5.97-5.68 (br. s, 1H), 5.45-5.20 (br. s, 2H), 4.55 (s, 2H), 4.33-4.27 (m, 2H), 4.27-4.21 (m, 2H), 4.15 (d, J=8 Hz, 2H), 3.85-3.77 (m, 2H), 3.74-3.58 (m, 20H), 3.58-3.47 (m, 8H), 3.41-3.33 (m, 4H), 3.33-3.25 (m, 2H), 2.35-2.16 (m, 12H), 1.76-1.46 (m, 4H), 1.35 (quintet, J=8.7 Hz, 2H), 1.01-0.88 (m, 4H). UPLC-MS (ESI+) calculated for C₅₂H₇₃F₅N₅O₁₉S⁺(M+H)⁺ 1198.45 found 1198.77.

### Example 17. Synthesis of Linker Drug Construct 16 by Coupling of Activated Ester 14 with va-PABC-PBD

To a solution of deprotected PBD diner (12.6 mg, 13.7 μmol, 1 equiv.) in 4 mL dry CH₂Cl₂ was added a solution of compound 14 (25.8 mg, 21.5 μmol, 1.58 equiv.) in 1 mL dry CH₂Cl₂ containing Et₃N (5.71 μL, 41.0 μmol, 3 equiv.). The resulting mixture was wrapped in aluminum foil and stirred for 15.5 hours at room temperature. UPLC-MS analysis showed a conversion of 67% and still the presence of H₂N-VA-PABC-PBD. The reaction mixture was concentrated to about 2 mL volume and additional compound 14 (21.4 mg, 17.9 μmol; 1.3 equiv.) in 300 μL dry CH₂Cl₂ was added. Stirring was continued at room temperature for an additional 4 hours after which UPLC-MS analysis showed a conversion of 93% and trace amounts of H₂N-VA-PABC-PBD. The reaction mixture was subsequently concentrated in vacuo at 25° C. and the residue was purified by A.F.C.C. (Sepacore); 4 G Premium; flow 10 ml/min; 3 ml per fraction; runtime minutes; 0%→10% CH₃OH/CH₂Cl₂; Fractions containing product were combined and concentrated in vacuo to yield construct 16 as a colorless film (15.7 mg, 8.11 μmol; 56%). UPLC-MS (ESI+) calculated for C₁₀₄H₁₆₉N₁₇O₃₄S₃+(M+H)⁺ 1936.89 found 1936.95.

### Example 18. Synthesis of Compound 17 by CSI Reaction of Diamine 10

A solution of BCN-OH (0.32 g, 2.1 mmol, 2.5 equiv.) in dry CH₂Cl₂ (5 mL) was treated with CSI (177 μL, 2.0 mmol, 2.4 equiv.) and stirred at room temperature for 10 minutes. Nect, Et₃N (592 μL, 4.25 mmol, 5 equiv.) was added and after stirring for 8 minutes a solution of diamine 10 (412 mg, 0.85 mmol) in 6 mL dry CH₂Cl₂ (this solution was pre-treated with triethylamine (615 μL, 4.43 mmol, 5 equiv.) was added. The resulting reaction mixture was stirred for 21 hours at room temperature. The reaction mixture was concentrated in vacuo and the residue was purified by silicagel chromatography (0%→6% MeOH in CH₂Cl₂). The desired product was obtained as a pale yellow oil (321.7 mg, 0.35 mmol; 41%). ¹H NMR (400 MHz, CDCl₃+few drops CD₃OD) 5 (ppm) 4.34-4.20 (m, 6H), 3.76-3.68 (m, 4H), 3.68-3.57 (m, 18H), 3.57-3.49 (m, 4H), 3.34-3.23 (m, 4H), 2.37-2.14 (m, 12H), 1.66-1.49 (m, 4H), 1.40 (quintet, J=8.7 Hz, 2H), 1.04-0.92 (m, 4H).

### Example 19. Synthesis of Compound 18 by CSI Reaction of Alcohol Compound 17

To a solution of compound 17 (0.29 g, 0.31 mmol) in dry CH₂Cl₂ (6 mL) was added CSI (27 μL, 0.31 mmol, 1 equiv.). After stirring for 7 minutes TLC (5% MeOH in CH₂Cl₂) showed some starting material left. Additional CSI (5.4 μL, 62.9 μmol, 0.2 equiv.) was added and the mixture was stirred for another 8 minutes. Then Et₃N (131 μL, 0.94 mmol, 3 equiv.) was added and after stirring for 7 minutes a solution of methyl 2-(2-(2-aminoethoxy)ethoxy)acetate,HCl (87.4 mg, 0.41 mmol; 1.3 equiv.) in 2 mL dry CH₂Cl₂ (this solution was pre-treated with Et₃N (114 μL, 0.82 mmol, 2 equiv.) was added. The resulting mixture was stirred for 14 hours at room temperature. The reaction mixture was concentrated in vacuo and the residue was purified by silicagel chromatography (0%→5% MeOH in CH₂Cl₂). The desired product was obtained as a pale yellow oil (51.2 mg, 62.9 μmol; 13%). ¹H NMR (400 MHz, CDCl₃) δ (ppm) 4.35-4.20 (m, 8H), 3.79-3.75 (s, 3H), 3.75-3.59 (m, 26H), 3.59-3.52 (m, 4H), 3.40-3.27 (m, 6H), 2.37-2.17 (m, 12H), 1.66-1.49 (m, 4H), 1.40 (quintet, J=8.7 Hz, 2H), 1.04-0.92 (m, 4H). UPLC-MS (ESI+) calculated for C₄₇H₇₈N₇O₂₃S₃+(M+H)*1204.43 found 1204.62.

### Example 20. Synthesis of Carboxylic Acid 19 by Hydrolysis of Methyl Ester 18

To a turbid solution of compound 18 (51.2 mg, 62.9 μmol) in a 1:1 mixture of H₂O (500 μL) and THF (500 μL) was added an aqueous NaOH solution (195 μL, 195 μmol, 1M, 5 equiv.).

After stirring the mixture for 2.5 hours UPLC-MS showed full conversion to the product. The reaction mixture was poured into EtOAc (6 mL) and H₂O (6 mL). The pH of the water layer was adjusted to 3.5-4 with an aqueous HCl solution (1M). After separation of the layers the aqueous layer was extracted once more with EtOAc (6 mL). The organic layers were combined and dried over Na₂SO₄ and concentrated in vacuo. The residue was co-evaporated once with MeCN. The desired product was obtained as pale yellow film (36.9 mg, 31.0 μmol; 79%) and used without further purification in the next step. UPLC-MS (ESI+) calculated for C₄₆H₇₆N₇O₂₃S₃+(M+H)⁺ 1190.41 found 1190.70.

### Example 21. Synthesis of PFP-Ester 20 by Activation of Carboxylic Acid 19

To a solution of compound 19 (36.9 mg, 31.0 μmol) in 2 mL CH₂Cl₂ was added EDC.HCl (6.5 mg, 34 μmol, 1.1 equiv.). Next, a stock solution of pentafluorophenol (25 mg in 1 mL CH₂Cl₂) was prepared of which 205 μL (5.1 mg, 27.1 μmol, 0.9 equiv.) was added to the reaction mixture.

After stirring the mixture for 6.5 hours at room temperature, UPLC-MS showed still presence of some starting material. Additional EDC.HCl (1.2 mg, 6.2 μmol, 0.2 equiv.) and pentafluorophenol stock solution (46 μL, 6.2 μmol, 0.2 equiv.) were added and the mixture was stirred for another 15.5 hours. The reaction mixture was concentrated and the residue was purified by silicagel chromatography (first 0%→70% EtOAc in heptane followed by 0%→100% acetone in CH₂Cl₂). The desired product was obtained as a colorless film (4.0 mg, 2.9 μmol; 10%). UPLC-MS (ESI+) calculated for C₅₂H₇₅F₅N₇O₂₃S₃+(M+H)⁺ 1356.40 found 1356.56.

### Example 22. Synthesis of Linker Drug Construct 21 by Coupling of Activated Ester 20 with vc-PABC-MMAE

To a solution of H₂N—VC-PABC-MMAE.TFA in dry DMF (36.5 μL, 3.65 mg, 3.0 μmol) was added Et₃N (1.2 μL, 8.8 μmol, 3 equiv.). The resulting mixture was added to compound 20 (4.0 mg, 2.9 μmol) followed by addition of an extra 90 μL dry DMF. The mixture was reacted for 18 hours at room temperature. UPLC-MS showed clear product formation. The mixture was diluted with DMF to 420 μL and purified by RP-HPLC (C18, 30%→90% MeCN (1% AcOH) in water (1% AcOH). The desired product was obtained as a colorless film (3.1 mg, 1.4 μmol; 46%). UPLC-MS (ESI+) calculated for C₁₀₄H₁₆₉N₁₇O₃₄S₃²⁺ (M+2H)²+1148.56 found 1148.89.

1. A method for the preparation of an alkyne-linker-payload construct of structure Q-L-C(O)—NR³—D (1), comprising reacting:
(i) an alkyne compound of structure Q-L-C(O)-X (2), wherein
Q is an alkyne moiety selected from the group consisting of terminal alkyne and (hetero)cycloalkyne,
L is a linker, and
X is a leaving group selected from halogen, SR¹, O-succinimidyl, O-(hetero)aryl-(R²)₁₋₅, wherein R¹ is selected from C₁-C₆ alkyl and (hetero)aryl; and R² is C₁-C₆ alkyl, halogen or NO₂;
with
(ii) a molecule of structure D-NHR³ (3), wherein
D is a payload, and
R³ is selected from hydrogen, optionally substituted C₁-C₂₄ alkyl, optionally substituted aryl.

2. The method according to claim 1, wherein the payload D is a cytotoxin.

3. The method according to claim 1, wherein the cytotoxin is selected from the group consisting of taxanes, anthracyclines, camptothecins, epothilones, mytomycins, combretastatins, vinca alkaloids, maytansinoids, enediynes, duocarmycins, tubulysins, amatoxins, auristatins, pyrrolobenzodiazepines (or dimers thereof), indolino-benzodiazepines (or dimers thereof), isoquinolino-benzodiazepines (or dimers thereof), amanitins, ligands for radioisotopes, therapeutic peptides (or fragments thereof), kinase inhibitors, MEK inhibitors, KSP inhibitors, Bcl inhibitors, NAMPT inhibitors, PARP inhibitors and analogues or combinations or prodrugs thereof.

4. The method according to claim 1, wherein the (hetero)cycloalkynyl moiety Q is selected from the group consisting of (Q22)-(Q35c):

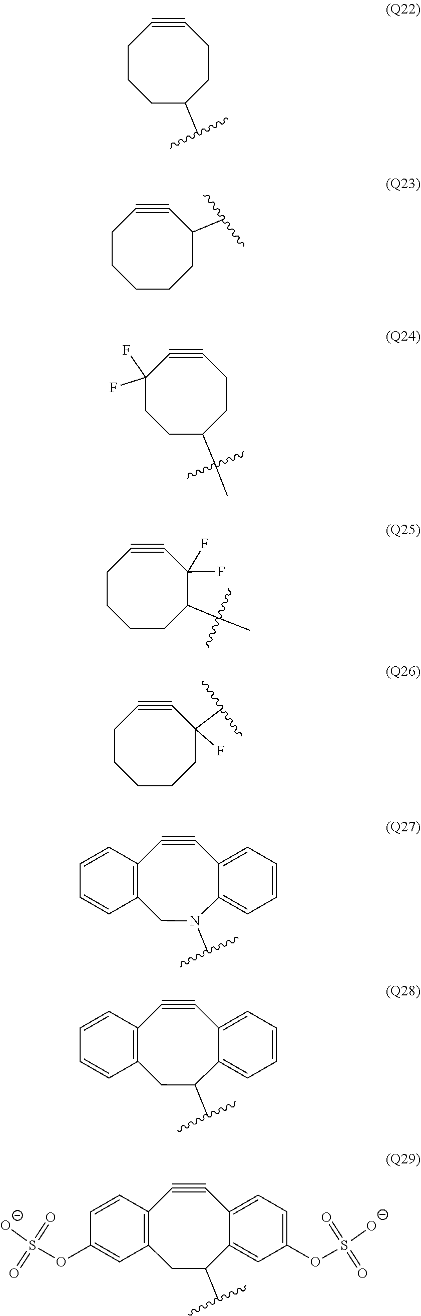

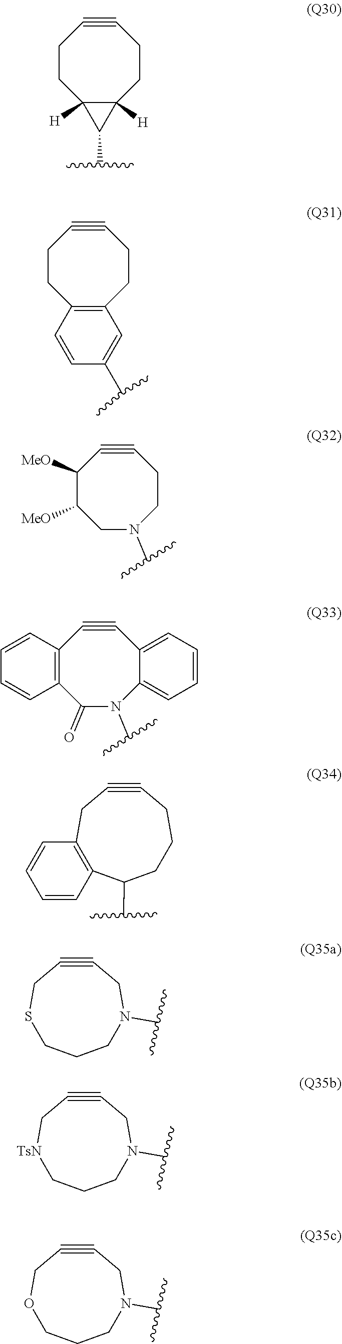

5. The method according to claim 1, wherein the (hetero)cycloalkynyl moiety Q is according to structure (Q36): 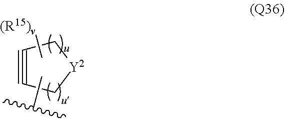
wherein:
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₆-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y² is C(R³¹)₂, O, S or NR³¹, wherein each R³¹ is individually is R¹⁵ or -(L¹)ₙ-(L2)ₒ-(L3)-(L⁴)_{q}-D;
u is 0, 1, 2, 3, 4 or 5;
u′ is 0, 1, 2, 3, 4 or 5, wherein u+u′=4, 5, 6, 7 or 8;
v=an integer in the range 8-16.

6. The method according to claim 1, wherein the cyclooctynyl moiety Q is according to structure (Q37): 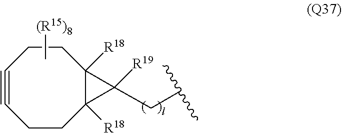
wherein
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁸ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
R¹⁹ is selected from the group consisting of hydrogen, -(L′)ₙ-(L²)ₒ-(L³)ₚ-(L⁴)_{q}-D; halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups, the alkyl groups optionally being interrupted by one of more hetero-atoms selected from the group consisting of O, N and S, wherein the alkyl groups, (hetero)aryl groups, alkyl(hetero)aryl groups and (hetero)arylalkyl groups are independently optionally substituted; and
l is an integer in the range 0 to 10.

7. The method according to claim 1, wherein the (hetero)cyclooctynyl moiety Q is according to structure (Q38): 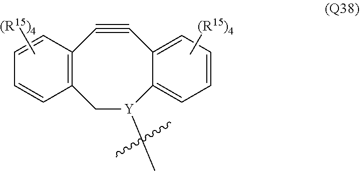
wherein
R¹⁵ is independently selected from the group consisting of hydrogen, halogen, —OR¹⁶, —NO₂, —CN, —S(O)₂R¹⁶, —S(O)₃⁽⁻⁾, C₁-C₂₄ alkyl groups, C₅-C₂₄ (hetero)aryl groups, C₇-C₂₄ alkyl(hetero)aryl groups and C₇-C₂₄ (hetero)arylalkyl groups and wherein the alkyl groups, (hetero)aryl groups, alkyl(hetero)aryl groups and (hetero)arylalkyl groups are optionally substituted, wherein two substituents R¹⁵ may be linked together to form an optionally substituted annulated cycloalkyl or an optionally substituted annulated (hetero)arene substituent, and wherein R¹⁶ is independently selected from the group consisting of hydrogen, halogen, C₁-C₂₄ alkyl groups, C₆-C₂₄ (hetero)aryl groups, C₇-C₂₄ alkyl(hetero)aryl groups and C₇-C₂₄ (hetero)arylalkyl groups;
Y is N or CR¹⁵.

8. The method according to claim 1, wherein linker L comprises a sulfamide group according to structure (L1) 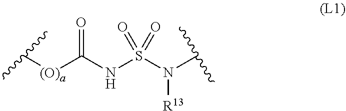
wherein
a=0 or 1, and
R¹³ is selected from the group consisting of hydrogen, C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups optionally substituted and optionally interrupted by one or more heteroatoms selected from O, S and NR¹⁴ wherein R¹⁴ is independently selected from the group consisting of hydrogen and C₁-C₄ alkyl groups, or R¹³ is a second occurrence of C(O)X connected to N via a spacer moiety.

9. The method according to claim 1, wherein linker L or payload D comprises a dipeptide or tripeptide spacer and/or a self-cleavable spacer.

10. The method according to claim 9, wherein the peptide spacer is selected from Val-Cit, Val-Ala, Val-Lys, Val-Arg, Phe-Cit, Phe-Ala, Phe-Lys, Phe-Arg, Ala-Lys, Leu-Cit, Ile-Cit, Trp-Cit, Ala-Ala-Asn and Ala-Asn.

11. The method according to claim 9, wherein the self-cleavable spacer is a para-aminobenzyloxycarbonyl according to structure (L4). 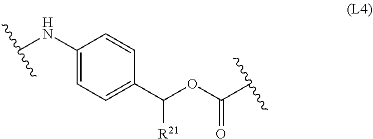
wherein:
R²¹ is H, R²² or C(O)R²², wherein R²² is C₁-C₂₄ (hetero)alkyl groups, C₃-C₁₀ (hetero)cycloalkyl groups, C₂-C₁₀ (hetero)aryl groups, C₃-C₁₀ alkyl(hetero)aryl groups and C₃-C₁₀ (hetero)arylalkyl groups, which optionally substituted and optionally interrupted by one or more heteroatoms selected from O, S and NR²³ wherein R²³ is independently selected from the group consisting of hydrogen and C₁-C₄ alkyl groups.

12. The method according to claim 11, wherein R²² is C₃-C₁₀ (hetero)cycloalkyl or polyalkylene glycol.

13. The method according to claim 1, wherein X is selected from halogen, O-succinimide, O-benzotriazole, 4-nitrophenol, 2,3,5,6-tetrafluorophenol, 2,3,4,5,6-pentafluorophenol or 2,3,4,5,6-pentachlorophenol.

14. The method according to claim 1, wherein the alkyne compound of structure (2) is prepared by activation of Q-L-C(O)OH.

15. A method for preparing a bioconjugate, comprising:
(a) preparing a cycloalkyne-linker-payload construct of structure Q-L-C(O)—NR³—D (1) using the method according to claim 1; and
(b) reacting the cycloalkyne-linker-payload construct with a biomolecule containing a moiety that is reactive towards an alkyne in a cycloaddition, to form a bioconjugate wherein the payload is covalently attached to the biomolecule.

16. The method according to claim 15, wherein the biomolecule is selected from the group consisting of proteins, polypeptides, peptides, glycans, lipids, nucleic acids, oligonucleotides, polysaccharides, oligosaccharides, enzymes, hormones, amino acids and monosaccharides.

17. The method according to claim 15, wherein the reaction (b) is a 1,3-dipolar cycloaddition or a [4+2] cycloaddition.

18. The method according to any one of claim 15, wherein moiety that is reactive towards an alkyne is selected from azide, tetrazine, triazine, nitrone, nitrile oxide, nitrile imine, diazo compound, ortho-quinone, dioxothiophene and sydnone.

19. An alkyne compound of structure Q-L-C(O)-X (2), wherein
Q is an alkyne moiety selected from the group consisting of terminal alkyne and (hetero)cycloalkyne,
L is a linker, and
X is a leaving group selected from halogen, SR¹, O-succinimidyl, O-(hetero)aryl(R²)₁₋₅, wherein R¹ is selected from C₁-C₆ alkyl and (hetero)aryl; and R²is C₁-C₆ alkyl, halogen or NO₂.
